(19) United States
(12) Patent Application Publication
(10) Pub. No.: US 2012/0083861 A1
(43) Pub. Date: Apr. 5, 2012

(54) SELECTIVE ACTIVATION OF NEURONS BY SINUSOIDAL ELECTRIC STIMULATION

(75) Inventors: Shelley I. Fried, Cambridge, MA (US); Daniel K. Freeman, Somerville, MA (US)

(73) Assignee: THE GENERAL HOSPITAL CORPORATION, Boston, MA (US)

(21) Appl. No.: 13/252,499

(22) Filed: Oct. 4, 2011

Related U.S. Application Data
(60) Provisional application No. 61/389,374, filed on Oct. 4, 2010.

(51) Int. Cl. A61N 1/36

(52) U.S. Cl. 607/76

(57) ABSTRACT

The present invention provides for a method of selectively activating synaptically mediated responses in ganglion cells without activating passing axons, by contacting a focal region around said cells with an electrode that stimulates using low-frequency sinusoidal electric signal. In particular, the selective low-frequency sinusoidal stimulation has a frequency of ≦25 Hz. specific frequencies of sinusoidal stimulation, which can be used to preferentially activate certain neural cell types, including retinal cells: ganglion cells at 100 Hz, photoreceptors are activated at 5 Hz, and bipolar cells at 25 Hz.

### CROSS-REFERENCE TO RELATED APPLICATIONS

This application claims the priority benefit of U.S. Provisional Application No. 61/389,374, filed Oct. 4, 2010, entitled A Method for Focal Activation of Neurons with Electrical Stimulation, incorporated entirely herein by this reference.

### FEDERAL FUNDING

This invention was made with federal funding under Career Development Awards (CDA-1), awarded by the Department of Veterans Affairs, and with Grant No. R01 EY-019967-01, awarded by the National Eye Institute. The U.S. Government has certain rights in the invention.

### BACKGROUND

Electric stimulation of the central nervous system (CNS) is being evaluated as a treatment modality for a variety of neurological, psychiatric, and sensory disorders. The remarkable successes of cochlear implants and deep brain stimulation (DBS) for the treatment of Parkinson's disease suggest a wide range of neurological disorders could also be treated with electric stimulation from a neural prosthetic. Clinical trials are underway targeting epilepsy, cluster headaches, depression, certain types of blindness, and other diseases of the CNS. Despite considerable effort, however, the outcomes many of these applications remain limited, in part, because these neural prostheses use electric stimulation with pulse trains to modulate neural activity, and pulse technology lacks fine control over the pattern of elicited activity. For example, in retinal prostheses the incidental stimulation of axons on the retinal surface diminishes the fidelity over the spatial pattern of activation. In addition, the temporal resolution of elicited spike trains through activation of the synaptic network with pulsatile stimulation has been quite limited. Improved stimulation methods that selectively activate individual classes of neurons or target specific neuronal substructures would be a significant benefit to neural prostheses.

### SUMMARY

The present invention provides for specific frequencies of sinusoidal stimulation, which can be used to preferentially activate certain neural cell types, including retinal cells: ganglion cells at 100 Hz, photoreceptors are activated at 5 Hz, and bipolar cells at 25 Hz. In addition, low-frequency stimulation (e.g., ≦25 Hz) did not activate passing axons but still elicited robust synaptically mediated responses in ganglion cells, and therefore elicited neural activity is confined to within a focal region around the stimulating electrode. The present invention provides for low-frequency sinusoidal stimulation that has significantly improved control over elicited neural activity relative to conventional pulsatile stimulation. This indicates that such stimulation can be used to restrict activity to only a small region close to the stimulating electrode. Activation of only those neurons close to the stimulating electrode does not activate axonal processes. This technique can be used in the retina as well as in a wide range of other neural stimulation applications.

Some embodiments of the present invention provide for a method of selectively activating synaptically mediated responses in ganglion cells without activating passing axons, by contacting a focal region around said cells with an electrode that stimulates using low-frequency sinusoidal electric signal. In particular, the selective low-frequency sinusoidal stimulation has a frequency of about ≦100 Hz, for example, ≦50 Hz, ≦30 Hz, ≦25 Hz.

An embodiment of the present invention provides for a method of selectively activating ganglion cells comprising exposing said ganglion cells to a sinusoidal electric signal stimulus of about 100 Hz. Another embodiment provides for a method of selectively activating photoreceptor cells comprising exposing said photoreceptor cells to a sinusoidal electric signal of about 5 Hz. Yet another embodiment provides for a method of selectively activating bipolar cells comprising exposing said bipolar cells to a sinusoidal electric signal of about 25 Hz.

### DESCRIPTION OF THE DRAWINGS

FIG. 1 is a schematic of the experimental setup. FIG. 1a: Spikes were recorded from a single ganglion cell (gray) using cell-attached patch clamping (patch electrode is orange). Epi-retinal stimulation is applied for two different positions of the stimulating electrode: the ‘Soma Position’ is centered over the sodium channel band (˜40 μm lateral from the soma) and the ‘Distal Axon Position’ is ˜1 mm lateral from the soma. Both locations are 25 μm above the inner limiting membrane. FIG. 1b: Sample of a response to 10-Hz stimulation where large spikes (arrows) are easily discerned from the sinusoidal stimulus artifact. FIGS. 1c, 1d: The number of spikes elicited in response to a 1-sec sinusoidal stimulus of 25 Hz (FIGS. 1c) and 100 Hz (FIG. 1d) as a function of stimulating electrode position for two different cells. The soma is defined as zero on the x-axis and the negative positions are distance from the center of the soma along the axon. Stimulus amplitudes used were 8, 11, 14, and 17 μA for 25 Hz, and 9, 12, and 15 μA for 100 Hz, with increasing amplitudes indicated by circle, square, triangle, and diamond.

FIG. 2 demonstrates avoiding axonal activation with low-frequency sinusoidal stimulation. FIG. 2a: The number of spikes elicited in response to a 1-sec, 10-Hz sinusoidal stimulus is plotted as a function of stimulus amplitude (peak-to-peak μA) for stimuli delivered near the soma (filled circles) and the distal axon (open circles). FIG. 2b-2c: Similar plots for stimulus frequencies of 25 and 100 Hz. FIG. 2d: The probability of eliciting a spike in response to 0.2-ms cathodal pulses (15-30 repeats at each stimulus level). All data in FIG. 2a-2d are from a single cell. FIG. 2e: Summary data showing the mean ratio of distal axon threshold to soma threshold for each stimulus waveform (n=10 cells per waveform). The upward-pointing arrows indicate values presented are lower bounds (see text). Error bars indicate standard error.

FIG. 3 shows that input from presynaptic neurons underlies the response to LFSS. FIG. 3a-3d: The response to sinusoidal stimulation (10 Hz, 25 Hz, 100 Hz) and 0.2-ms cathodal pulses delivered near the soma for control (circles), CNQX (squares), and CNQX+CdCl₂ (triangles). For sinusoidal stimulation, the number of spikes elicited in response to a 1-sec stimulus is plotted as a function of stimulus amplitude (peak-to-peak μA). In response to pulses, the probability of eliciting a spike is plotted against stimulus amplitude (15-30 repeats at each stimulus level). All data in FIG. 3a-3d are from a single cell. Legend in FIG. 3c applies to FIGS. 3a, 3b and 3d.

FIG. 4 shows whole-cell patch clamp reveals synaptic currents. Whole-cell voltage-clamping an OFF-cell at −60 mV reveals excitatory currents in response to 5-Hz sinusoidal stimulation for 1 sec. By convention, inward currents are depicted as negative deflections. Note that the sinusoidal stimulus artifact is also embedded in the response. The artifact is a zero-mean signal, however, and the fact that the current is skewed negatively is indicative of a stimulus-induced inward current.

FIG. 5 illustrates the effects of pharmacological blockers on threshold and maximal response. The ratio of stimulus thresholds measured across all cells in CNQX (FIG. 5a) (n=6) or CdCl₂ (FIG. 5b) (n=4) relative to control for 10 Hz, 25 Hz, 100 Hz, and 0.2 ms cathodal pulses (error bars indicate standard error; arrows indicate bar is lower bound). The maximum number of spikes elicited in response to 1-sec of sinusoidal stimulation in control conditions (no drugs) is plotted vs. the maximum number of spikes in CNQX (FIG. 5c) and CdCl₂ (FIG. 5d) for all cells. Circles: 10 Hz; triangles: 25 Hz; squares: 100 Hz.

FIG. 6 demonstrates that stimulus frequency alters the response phase. Portion of the response to 1 sec sinusoidal stimulus delivered near the soma at 5 Hz (FIG. 6a-6b) and 25 Hz (FIG. 6c-6d). At 5 Hz, spikes occurred during the peak of the sinewave (cathodal phase) for OFF cells and the trough of the sinewave (anodal phase) for ON cells. At 25 Hz, spikes occurred during cathodal phase for both cell types. FIG. 6e: Summary plot of stimulus frequency vs. the average phase at which the peak spiking response occurs for ON cells (squares, n=6) and OFF cells (circles, n=6). Note that the ON and OFF phase difference occurs at low but not high frequencies. Error bars indicate standard error.

FIG. 7 depicts a model sodium channel and two calcium channels respond optimally to different stimulus frequencies. Ten voltage steps were made starting from −80 mV in steps of 10 mV (FIG. 7a) and the resulting L-type calcium (FIG. 7b) and sodium (FIG. 7c) currents were computed. Voltage was sinusoidally varied around −80 mV at 10 Hz and 200 Hz (FIG. 7d) (peak-to-peak amplitude: 100 mV) and the resulting L- and T-type calcium and sodium currents were calculated. (FIG. 7g) The peak current for calcium and sodium was calculated for frequencies ranging from 1 Hz to 1000 Hz.

FIG. 8 shows that the highest sensitivity to sinusoidal stimulation is over the axonal sodium-channel band. The number of spikes elicited in response to a 1-sec sinusoidal stimulus of 25 Hz (FIGS. 8a) and 100 Hz (FIG. 8b) as a function of stimulating electrode position for two different cells. Position 0 is directly over the soma and the negative positions are distance from the center of the cell body along the axon. Because the bath included the synaptic blocker CdCl₂, all responses resulted from direct excitation of the ganglion cell. Notice the peak response sensitivity was measured on the axon ˜40 p.m from the soma, indicating that it is the dense band of sodium channels that is the most sensitive region for electric stimulation of 25 Hz and 100 Hz sinusoids. The stimulus amplitudes used were 8, 11, 14, and 17 μA for 25 Hz, and 9, 12, and 15 μA for 100 Hz, with increasing amplitudes indicated by circle, square, triangle, and diamond.

FIG. 9 shows a two-compartment, passive model of a bipolar cell. FIG. 9A: Morphological reconstruction of a rod bipolar cell illustrating the soma, axon, and terminal regions. FIG. 9B: Schematic of the two-compartment model. The soma and terminal region are each represented by a resistor and capacitor in parallel. The resistance to intra-axonal current flow is represented by a resistor (Rₐₓₒₙ). The stimulus is represented by a voltage applied extracellularly across the soma and terminal compartments (Vₛₜᵢₘ). C. The transfer function (Vₜₑᵣₘ/Vₛₜᵢₘ) was normalized and plotted versus stimulus frequency, where Vₜₑᵣₘ represents the membrane potential at the terminal compartment. The cutoff frequency (895 Hz) was defined as the frequency at which Vₜₑᵣₘ/Vₛₜᵢₘ was reduced to 3 dB. The nominal values of the circuit elements were: Rₛₒₘₐ=5.98 GΩ, Cₛₒₘₐ=3.7 pF, Rₜₑᵣₘ=27.9 GΩ, Cₜₑᵣₘ=0.8 pF, Rₐₓₒₙ=272.2 MΩ.

FIG. 10 demonstrates changes to axonal resistance alter the cutoff frequency. FIG. 10A: The transfer function of the two-compartment model is shown for the nominal value of Rₐₓₒₙ (272.2 MΩ), as well as for one-half and double this value. The nominal curve was normalized to unity and the other curves are scaled relative to this value. FIG. 10B: The cutoff frequency is plotted for values of Rₐₓₒₙ ranging from ⅛ to 8× nominal; the arrow indicates the nominal value.

FIG. 11 illustrates the effect of varying the resistance and capacitance of the soma and terminal compartments on the transfer function of the two-compartment model. The effect of varying Rₛₒₘₐ (FIG. 11A), Rₜₑᵣₘ (FIG. 11B), Cₛₒₘₐ (FIG. 11C) and Cₜₑᵣₘ (FIG. 11D) on the transfer function. Variations in the size of the soma (FIG. 11E) and terminals (FIG. 11F) were simulated by varying resistance and capacitance simultaneously. For all plots, the nominal curve was normalized to unity and the other curves were scaled relative to this value (see Methods). The legend in FIG. 11A applies to all plots.

FIG. 12 shows a multi-compartment, passive model of a bipolar cell. FIG. 12A: The stimulus is represented by a point source that was positioned 40 μm from the terminals. During stimulation, the extracellular voltage (Vₑ) is computed as a function of distance, r, from the point source. FIG. 12B: Schematic of the multi-compartment model. Each compartment contains a resistor and capacitor in parallel, representing the leak conductance (gₗₑₐₖ) and membrane capacitance (Cₘ), respectively. Intracellular current flow between neighboring compartments is represented by conductance, gᵢₙₜᵣₐ. FIG. 12C: The frequency response was measured by sinusoidally modulating Vₑ(r) and measuring the resulting membrane potential in the terminals (Vₜₑᵣₘ). Nominal membrane parameters were Cₘ=1.07 μF/cm², gₗₑₐₖ=48.00 μs/cm², pᵢ=189.6 Ωcm.

FIG. 13 shows the effect of varying somatic and terminal membrane parameters on the frequency response of the multi-compartment model. The membrane potential in the terminals (Vₜₑᵣₘ) in response to sinusoidal stimulation is shown for variations in somatic (13A) and terminal (13B) membrane conductance (Gₗₑₐₖ), as well as somatic (13C) and terminal (13D) capacitance (Cₘ). The size of the soma (13E) and terminals (13F) was varied by scaling both conductance and capacitance. (i.e., doubling the size of the soma is simulated by doubling both gₗₑₐₖ and Cₘ in all the soma compartments). For all plots, the nominal curve was normalized to unity and the other curves were scaled relative to this value (see Methods). The legend in panel A applies to all plots.

FIG. 14 shows changes in intra-axonal resistance alter the cutoff frequency in the multi-compartment model. The membrane potential in the terminals (Vₜₑᵣₘ) is measured as a function of stimulus frequency for variations in axonal membrane capacitance (FIG. 14A) and conductance (FIG. 14B), axonal resistivity (FIG. 14C), and axonal diameter (FIG. 14E). The cutoff frequency is plotted as a function of total intra-axonal resistance (FIG. 14D). The arrow indicates the nominal resistance. For plots in FIG. 14A-14C and 14E, the nominal curve was normalized to unity and the other curves were scaled relative to this value (see Methods). The legend in FIG. 14A applies to FIGS. 14B, 14C, and 14E.

FIG. 15 shows the effect of varying axonal length and electrode distance on the frequency response of the multi-compartment model. FIG. 15A: Axonal length was changed to one-half and then twice that of nominal with the stimulating electrode at a fixed distance from the terminals (40 μm). FIG. 14B: Each trace in panel A was normalized to unity and re-plotted to allow comparison of the cutoff frequency. FIG. 14C: The distance of the stimulating electrode was changed to one-half and then twice that of the nominal value while the length of the axon was held constant. FIG. 14D: The traces in FIG. 14C were all normalized to unity and re-plotted. The legend in FIG. 14A applies to all plots. For FIGS. 14A and 14C, the nominal curve was normalized to unity and the other curves were scaled relative to this value.

FIG. 16 is the frequency response of L-type calcium channels changes with stimulus amplitude. FIG. 16A: Illustration of the model for L-type calcium channel. The current, I_{L}, is measured in response to sinusoidal modulations in voltage (V), and g_{L} is related nonlinearly to voltage. FIG. 16B: Peak-to-peak calcium current is measured as a function of frequency for voltage fluctuations ranging from 2.5 to 20 mV. (16C) The traces in FIG. 16B were re-plotted after normalizing all curves to unity. FIG. 16D: Same as in FIG. 16B, but for fluctuations in voltage ranging from 20 mV to 100 mV. For clarity, the trace obtained for the 20 mV stimulus in FIG. 16B was re-plotted. FIG. 16E: The peak-to-peak current is plotted as a function of stimulus voltage for 10 Hz and 200 Hz. FIG. 16F: The cutoff frequency is plotted as a function of stimulus amplitude.

FIG. 17 shows the frequency response of T-type calcium channels is bandpass. FIG. 17A: Illustration of the model for T-type calcium channel. The current, I_{T}, is measured in response to sinusoidal modulations in voltage (V), and g_{T} is related nonlinearly to voltage. FIG. 17B: The peak-to-peak calcium current was measured as a function of frequency for fluctuations in voltage ranging from 2.5 to 20 mV. (17C) Re-plotting the traces in FIG. 17B after normalizing all curves to unity. FIG. 17D: Same as in FIG. 17B, but for fluctuations in voltage ranging from 20 to 100 mV. For clarity, the trace obtained for the 20 mV stimulus in FIG. 17B was re-plotted. FIG. 17E: The peak-to-peak current is plotted as a function of stimulus voltage for 10 Hz and 200 Hz. FIG. 17F: The frequency at which I_{T} is maximal as a function of stimulus amplitude.

FIG. 18 illustrates the effect of stimulus frequency on the activation/inactivation variables of L- and T-type calcium channels. FIG. 18A: The T-type activation variable, n(t), is plotted for stimulus frequencies of 10 Hz (top) and 200 Hz (bottom). FIG. 18B: Similar plots for the L-type activation variable, m(t). FIG. 18C-18E: The peak-to-peak and mean response is shown as a function of frequency for the T-type activation variable, n(t), the L-type activation variable, m(t), and the T-type inactivation variable, h(t). FIG. 18F: The scaling factors used to compute channel conductance are plotted for T-type channels (n(t)*h(t)) (top) and L-type channels (m²(t)) (bottom). The plots in FIG. 18F were calculated using the mean value of each gating variable (i.e., the lower plots in FIG. 18C-18E). The stimulus amplitude was 40 mV in all cases.

FIG. 19 indicates that incorporating calcium channels to the multi-compartment model does not affect the shape of the frequency response. FIG. 19A: A single compartment of the multi-compartment model with calcium channels added. I_{L} and I_{T} represent current through the L- and T-type calcium channels, respectively. FIG. 19B: The membrane potential in the terminals (Vₜₑᵣₘ) was measured in response to extracellular sinusoidal stimulation (Vₑ(r)). The stimulus amplitude was adjusted to give peak modulations in Vₜₑᵣₘ of 5, 20, 40, and 100 mV. The frequency response for each stimulus amplitude was normalized to unity and plotted along with the response of the passive model (i.e. no calcium channels).

FIG. 20 shows that L- and T-type calcium channel dynamics limit the frequency response of calcium currents measured in the multi-compartment model. The peak-to-peak current through L-type (I_{L}) (FIGS. 20A, 20B) and T-type (I_{T}) (FIGS. 20D, 20E) channels in response to extracellular sinusoidal stimulation. The stimulus amplitude was adjusted to give peak modulations in membrane potential in the range of 2.5 to 100 mV, as indicated in the legends. The traces obtained for the 20 mV stimulus in FIGS. 20A and 20D have been re-plotted in FIGS. 20B and 20E, respectively. The cutoff frequency of the L-type current (FIG. 20C), and the peak frequency of the T-type current (FIG. 20F) are plotted versus stimulus amplitude.

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Electric stimulation of the CNS is being evaluated as a treatment modality for a variety of neurological, psychiatric, and sensory disorders. Despite considerable success in some applications, existing stimulation techniques offer little control over which cell types or neuronal substructures are activated by stimulation. The ability to more precisely control neuronal activation would likely improve the clinical outcomes associated with these applications. The present invention provides for specific frequencies of sinusoidal stimulation, which can be used to preferentially activate certain neurons and retinal cell types: photoreceptors are activated at 5 Hz, bipolar cells at 25 Hz, and ganglion cells at 100 Hz. In addition, low-frequency stimulation (≦25 Hz) did not activate passing axons but still elicited robust synaptically mediated responses in ganglion cells; therefore, elicited neural activity is confined to within a focal region around the stimulating electrode. The present invention provides for low-frequency sinusoidal stimulation that has significantly improved control over elicited neural activity relative to conventional pulsatile stimulation.

Moreover, because extracellular electric stimulation with sinusoidal waveforms allows preferential activation of individual types of retinal neurons by varying stimulus frequency, as shown herein, the mechanisms underlying this frequency dependence has been characterized herein as a step towards improving methods of preferential activation. These mechanisms were elucidated by implementing a morphologically realistic model of a retinal bipolar cell and measured the response to extracellular stimulation with sinusoidal waveforms. This compared the frequency response of a passive membrane model to the kinetics of voltage-gated calcium channels that mediate synaptic release. The passive electrical properties of the membrane exhibited lowpass filtering with a relatively high cutoff frequency (nominal value=717 Hz). The cutoff frequency was dependent on intra-axonal resistance, with shorter and wider axons yielding higher cutoff frequencies. The cutoff frequency of bipolar cell synaptic release was primarily limited, however, by the relatively slow opening kinetics of L- and T-type calcium channels. The cutoff frequency of calcium currents depended nonlinearly on stimulus amplitude, but remained lower than the cutoff frequency of the passive membrane model for a large range of membrane potential fluctuations. These results suggest that although it may be possible to modulate the membrane potential of bipolar cells over a wide range of stimulus frequencies, synaptic release will only be initiated at the lower end of this range.

The remarkable successes of cochlear implants (Wilson & Dorman, 45 J. Rehabil. Res. 695 (2008)), and deep brain stimulation (DBS) for the treatment of Parkinson's disease (Gale et al., 32 Neuroschi. Meths. 378 (2008)), suggest a wide range of neurological disorders could also be treated with electric stimulation from a neural prosthetic. Clinical trials are underway targeting epilepsy (Loddenkemper et al., 113 J. Clin. Neurophysiol. 1667 (2001)), cluster headaches (Sillay et al., 38 Neruol. Dis. 361 (2010)), depression (Stefurak et al., 18 Mov. Disord. 1508 (2003)), certain types of blindness (Jensen et al., 44 Invest. Ophthalmol. Vis. Sci. 3533 (2003)), and other CNS diseases. Despite considerable effort, however, the outcomes of many of these applications remain limited. Improved stimulation methods that selectively activate individual classes of neurons or target specific neuronal substructures would be a significant benefit to neural prostheses.

For example, diseases of the outer retina such as macular degeneration and retinitis pigmentosa result in degeneration of the photoreceptors, the neurons primarily responsible for sensing light. Many neurons in the inner retina, including bipolar and ganglion cells, remain viable. Strettoi et al., 43 Vis. Res. 867 (2003); Margolis et al., 28 J. Neurosci. 6526 (2008); Mazzoni et al., 28 J. Neurosci. 14282 (2008). Retinal prostheses aim to restore vision to those blinded by outer retinal diseases by electrically stimulating the surviving neurons in the inner retina. Zrenner, 216(S1) Ophthalmologica 8 (2002); Winter et al., 18 J. Biomat. Sci. Polym. Ed. 1031 (2007). Although electric stimulation of the retina in blind subjects typically elicits a visual percept (Humayun et al., 43 Viosn Res. 2573 (2003); Rizzo et al., 44 Invest. Ophthalmo. Vis. Sci. 5362 (2003)), the ability to elicit more complex pattern vision with multi-electrode stimulation has not yielded consistent results. Rizzo et al., 2003; Lowenstein, 122 Arch. Ophthalmol. 587 (2004); Weiland et al., Ann. Rev. Biomed. Engin. (2004); Caspi, 127 Arch. Ophthalmol. 398 (2009). The quality of elicited vision must be improved in order for such devices to significantly affect quality of life. Chader et al., 175 Prog. Brain Res. 317 (2009). Although several factors are thought to limit the quality of elicited vision, the inability to control the pattern of elicited neural activity is thought to play a critical role. Presumably, stimulation methods that could replicate one or more aspects of normal retinal signaling would lead to the highest quality of elicited vision.

One of the obstacles to improving the quality of vision with retinal prostheses is thought to be the inability to control the spatial and temporal pattern of elicited ganglion cell spike trains. The manner in which ganglion cells encode visual information under normal physiological conditions is thought to be complex (Field & Chichilnisky, 30 Ann. Rev. Neurosci. 1 (2007); Gollisch & Meister, 65 Neuron 150 (2010), suggesting that sophisticated stimulation methods may be needed to replicate such spiking patterns. Using electric stimulation, spiking can be elicited in the ganglion cells via direct activation of the ganglion cell, or indirectly, by activating presynaptic neurons (e.g., bipolar cells) and thereby altering the levels of synaptic release onto the ganglion cells. Jensen et al., 2 J. Neural Engin. S16 (2005a); Fried et al., 95 J. Neurophysiol. 970 (2006); Margalit & Thoreson, 47 Invest. Ophthalmol. Vis. Sci. 2606 (2006); Sekirnjak et al., 95 J. Neurophysiol. 3311 (2006); Freeman & Fried, 8 J. Neural Engin. 016008 (2011).

Thus, efforts to control the spatial pattern of neural activation in retinal explants have had only limited success. Greenberg, Biomed. Engin. (1998); Jensen et al., 2003; Behrend et al., 172 J. Neurosci. Meths. 166 (2009). This is thought to arise from the ganglion cell bodies that are the target of stimulation are overlaid by axons that arise from distant cell bodies, and because the threshold for activation of these passing axons is higher than that of the soma region, but only by a factor of 2. Jensen et al., 2003. For example, incidental stimulation of these passing axons will be perceived by the brain as coming from ganglion cells with distant cell bodies, thereby reducing the spatial control over the elicited visual percept. Given that the activation threshold varies for different types of ganglion cells (e.g., brisk-transient versus local edge detectors) (Fried et al., 101 J. Neurophysiol. 1972 (2009)), the ability to activate a large number of ganglion cells while avoiding the activation of passing axons may not be possible with existing stimulation methods. In other words, the ability to selectively or even preferentially activate the direct versus indirect response is limited using stimulation with pulse trains. Jensen et al., 46 Invest, Ophthalmol. Vis. Sci. 1486 (2005b); Fried et al., 2006; Tsai et al., 102 J. Neurophysiol. 2982 (2009); but see Stett et al., 4 J. Neural Engin. S7 (2007).

A similar challenge exists in many other CNS-based neural prosthetic applications since targeted cell bodies often lie in close proximity to passing axons that arise from distant regions of the brain. Ranck, 98 Brain Res. 417 (1975); Jensen et al., 2003; Schiefer & Grill 14 IEEE Trans. Neural Sys. Rehab. Engin. 5 (2006); Behrend et al., 2009; Histel et al., 63 Neuron 508 (2009). For example, in DBS treatment of Parkinson's disease, the activation of passing axons in the limbic system is thought to underlie a number of adverse side effects, such as cognitive and mood changes. Wichmann & Delong, 52 Neuron 197 (2006).

The ability to selectively target individual classes of neurons would be another significant benefit to many neural prostheses. In the retina, selective activation of bipolar cells would utilize circuits in the inner retina, creating spiking patterns in ganglion cells that better resemble those that arise under physiological conditions. Bipolar cells can be activated by long-duration pulses (>1 ms) (Greenberg, 1998; Jensen et al., 2003; Jensen et al., 2005a; Fried et al., 2006), but such pulses also activate ganglion cells, both at the soma and the distal axon. This results in spiking patterns that are highly complex and do not resemble those that arise under physiological conditions. The ability to selectively activate particular classes of neurons could also be useful to many other neural prostheses (McIntyre & Grill, 88 J. Neurophysiol. 1592 (2002)), because stimulating electrodes are typically surrounded by heterogeneous populations of neurons.

The use of alternative stimulation waveforms (i.e., non-pulsatile) for electric stimulation have not been well explored. But see also Langille et al., 118 Intl. J. Neurosci. 1131 (2008); Cantrell & Troy, Conf. Proc. IEEE Ening. Med. Biol. Soc. 642 (2009)). This may be due, in part, to the early successes of pulsatile stimulation in cochlear implants and DBS for Parkinson's Disease. Given that the membrane properties of different neuronal substructures (e.g., soma versus axon) vary considerably in terms of the types and densities of voltage-gated ion channels, input resistance, capacitance, and synaptic contacts (Carras et al., 67 J. Neurophysiol. 292 (1992); O'Brien et al., 538 J. Physiol 787 (2002); Fried et al., 2009), such variability may lead to different frequency-dependent response properties for each substructure. This raises the possibility that the use of narrowband waveforms, such as sinusoids, may provide selective control over the targets of neuronal activation. Conversely, pulses contain broad spectral energy that may limit the ability to preferentially activate neuronal targets even if they exhibit different frequency-dependent properties.

Spike trains from rabbit retinal ganglion cells in response to sinusoidal electric stimulation of various frequencies (5-100 Hz) were measured and the responses compared to that of conventional pulse trains. Because of the well-defined organization of the retina, stimulation could be delivered near the soma as well as over the distal axon (˜1 mm from the soma) of the same cell, and the response for each location compared directly. Also, using pharmacological blockers, the components of the response due to direct activation of the ganglion cell, or due to activation of presynaptic neurons, were elucidated.

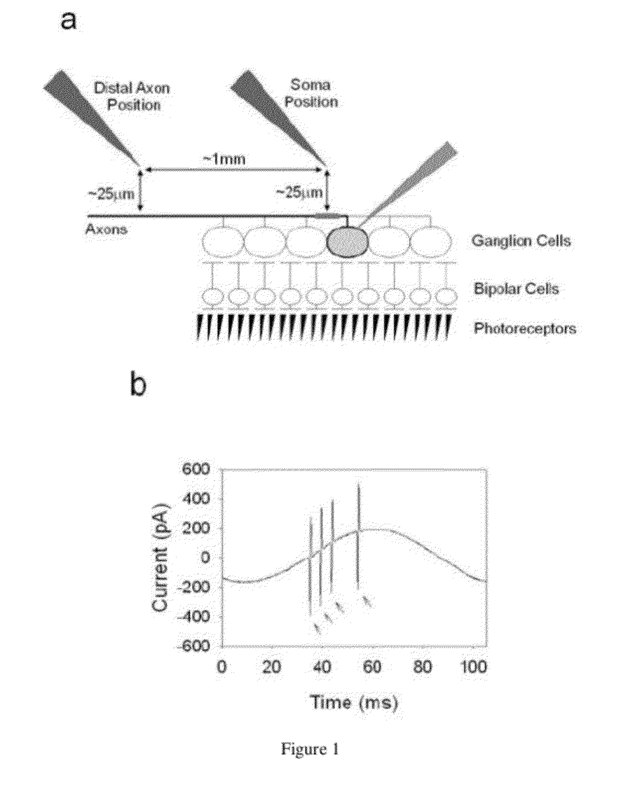

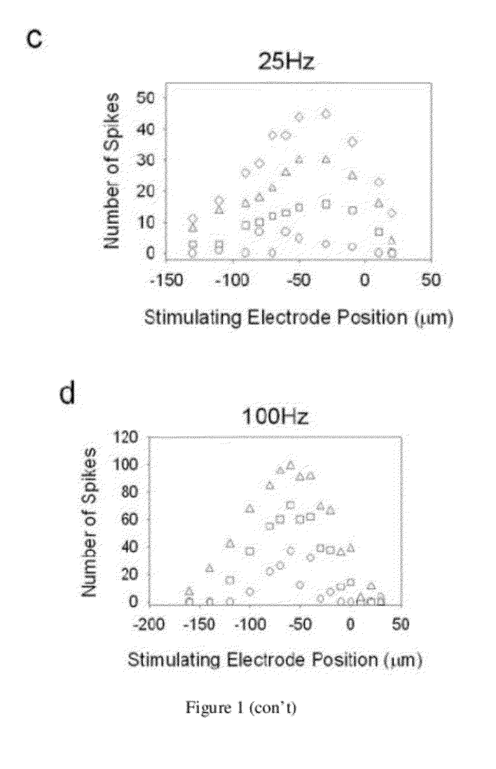

More specifically, cell-attached patch clamp recordings to measure spiking from retinal ganglion cells in response to electric stimulation with sinusoidal and pulsatile waveforms. Stimuli were delivered either in the soma region or over the distal axon (FIG. 1a). A typical response to one period of a 10-Hz stimulus delivered near the soma is shown in FIG. 1b—the use of patch clamp recordings allowed individual spikes (arrows) to be visualized without obstruction by the stimulus artifact. Previous work has shown that there is a dense band of sodium channels in the proximal axon (˜40 μm lateral from soma), and in response to pulses this region has the highest sensitivity to stimulation. Fried et al., 2009. This result was extended, herein, to include sinusoidal stimulation, where the maximal response to 25 and 100 Hz was found to occur ˜40-50 μm from the soma (FIG. 1c-1d) (n=3). For these preliminary experiments, synaptic input to the ganglion cell was blocked with application of CdCl2 (100 μM) in order to confirm that the response was mediated by direct activation of the ganglion cell and not activation of presynaptic neurons. All of the stimulation delivered near the soma in this study was approximately centered over the cell's sodium-channel band.

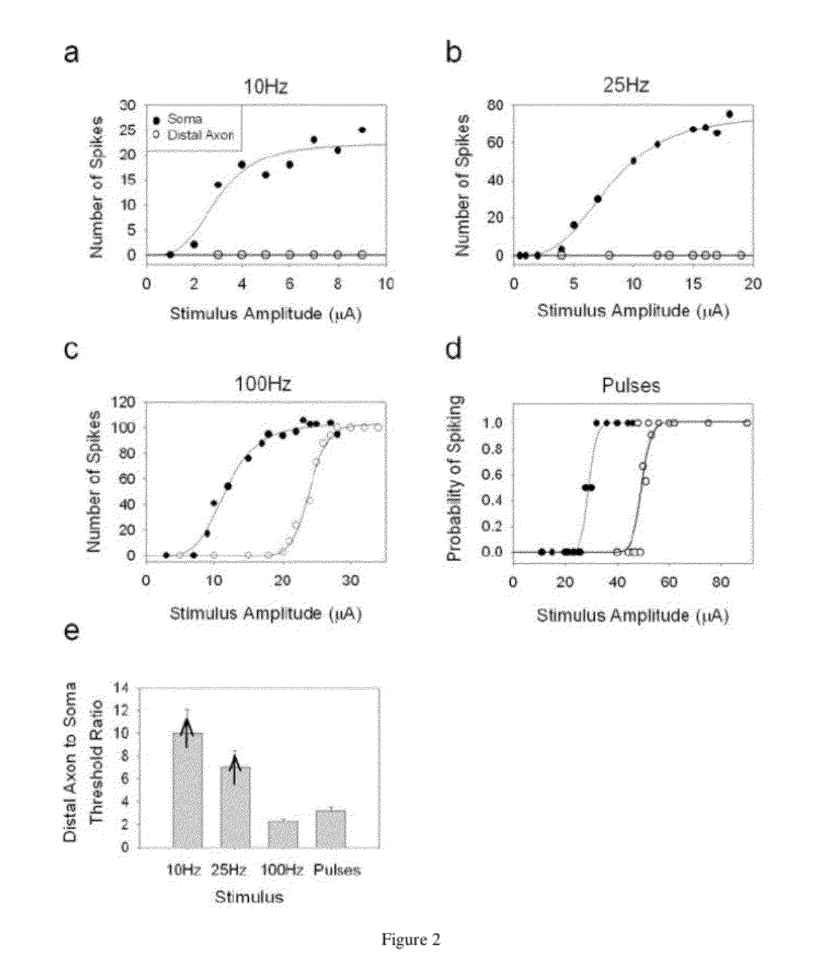

Avoiding axonal activation with sinusoidal stimuli was determined by comparing responses from electric stimuli delivered near the soma to responses from electric stimuli delivered over the distal axon, typically ˜1 mm from the soma (FIG. 1a). Stimulation waveforms consisted of: (1) low-frequency sinusoidal stimuli (LFSS) of 10 and 25 Hz, (2) high-frequency sinusoidal stimuli (HFSS) of 100 Hz, and (3) brief cathodic pulses of 0.2 ms delivered at 10 pulses per second. Sinusoidal stimulation of 10 Hz elicited a strong response when the stimulating electrode was positioned near the soma (FIG. 2a, filled circles), but spiking could not be elicited when the electrode was moved to a position over the distal axon (FIG. 2a, open circles) (n=10/10 cells). Even the highest amplitude levels, delivered safely, failed to elicit spiking at the distal axon position using 10-Hz stimulation. Similar results were obtained for stimulation at 25 Hz (FIG. 2b): cells were highly sensitive to stimulation near the soma, while stimulation over the distal axon elicited no spiking in most cases (n=9/10 cells) and elicited a response above threshold in only one cell (n=1/10).

Increasing the stimulus frequency to 100 Hz resulted in strong spiking responses, both when the stimulating electrode was positioned near the soma and also when it was positioned over the distal axon (FIG. 2c). Unlike LFSS, responses to 100 Hz typically consisted of a single spike per stimulus period resulting in a maximum of ˜100 spikes for a 1-sec stimulus. This maximum response level was reached for stimulation at both locations, although larger stimulus amplitudes were required when the stimulating electrode was over the axon (p<0.001, paired t-test). When short-duration (0.2 ms) cathodal pulses were applied, no more than a single spike per pulse was elicited. See Fried, 2006; Sekirnjak et al., 95 J. neuroophysiol 3311 (2006). For each stimulus amplitude, 15 to 30 pulses were delivered, and the number of pulses that elicited a spike was normalized to the total number of pulses delivered to give the fraction of pulses that elicited spikes (FIG. 2d).

These findings suggest that LFSS elicits a spiking response when the stimulating electrode is positioned near the soma but typically elicits no response when the stimulating electrode is positioned over the distal axon. HFSS and pulses elicit responses for both electrode positions. To quantify these results, the stimulus amplitude that was needed to elicit a given response level was computed ('threshold') at each of the two locations. The threshold ratios (distal axon/soma region) measured for HFSS and pulses were 2.29±0.07 and 3.22±0.08, respectively (FIG. 2e) (mean±standard error). The threshold ratios for LFSS were 10.0±0.66 for 10 Hz and 7.08±0.43 for 25 Hz. Because LFSS stimulation near the axon did not elicit spiking at the maximum level tested for 18/20 cells, the maximum amplitude tested as a lower bound of threshold and a lower bound on the distal axon-to-soma threshold ratios for 10 and 25-Hz stimulation (indicated by the arrows in FIG. 2e). The HFSS and pulse threshold ratios are each significantly smaller than each of the LFSS threshold ratios (maximum value of all comparisons, p<0.015). The relatively high threshold ratios for LFSS suggest that ganglion cells whose somas are close to the stimulating electrode will respond while nearby passing axons will not. Thus LFSS may be useful in confining elicited activity to a small, ‘focal’ region around the electrode.

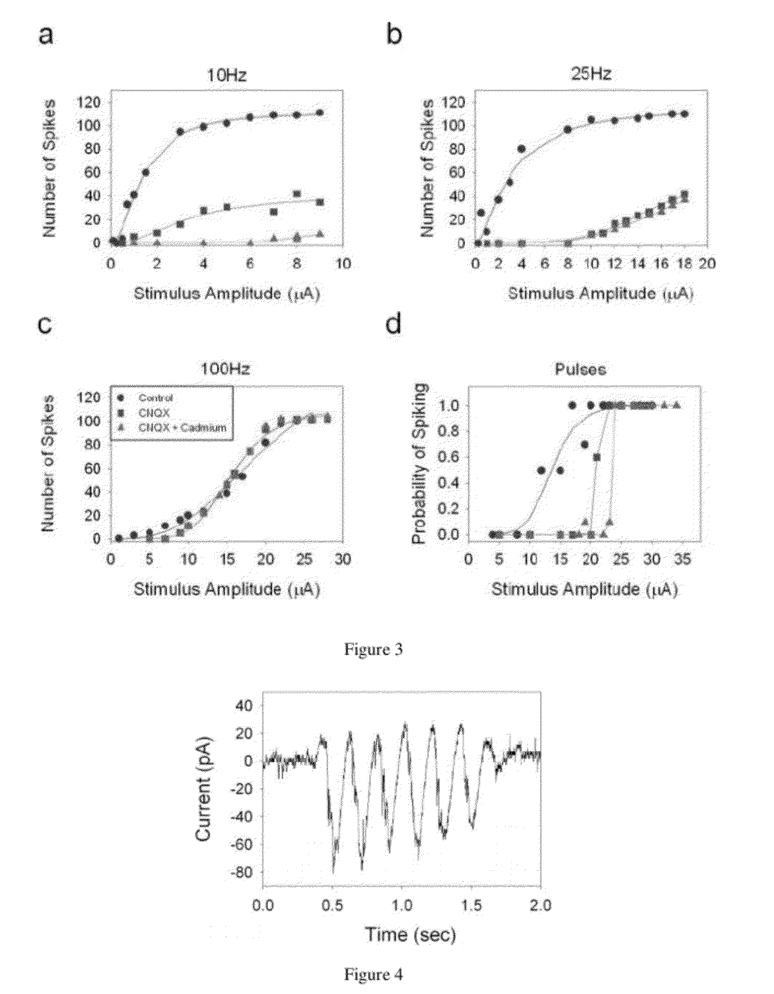

The present invention provides for responses to LFSS that are synaptic in origin. To determine whether presynaptic activation played a significant role in the high sensitivity of the soma region to LFSS, the response to stimulation near the soma was measured while synaptic transmission was blocked pharmacologically. The primary source of excitatory input to ganglion cells arises via glutamatergic release from the axon terminals of bipolar cells and is mediated through AMPA/kainate receptors on the ganglion cell dendrites. In the presence of 50 μM CNQX, an antagonist of AMPA/kainate receptors, the response to 10-Hz stimulation was greatly reduced (n=3/6) (FIGS. 3a) or completely eliminated (n=3/6). To determine whether the CNQX-insensitive portion of the response was mediated by one or more additional synaptic components, 100 μM CdCl₂ was added to block all synaptic transmission (Margalit & Thoreson, 2006), and found that now, the response was mostly eliminated (FIG. 3a) (n=2/2). Similar findings were obtained in CdCl₂ alone: the response to 10-Hz stimulation at the soma was completely eliminated (n=3/4) or greatly reduced (n=1/4). Taken together, these results suggest that the response to 10 Hz is primarily mediated through synaptic activity.

The amount of presynaptic activation was similarly determined for sinusoidal stimulation at 25 and 100 Hz, and with 0.2-ms pulses (FIGS. 3b-3d). The response to 25-Hz stimulation was greatly reduced in the presence of CNQX (n=6) or CdCl₂ (n=4) or both (n=2). Because the response to 25 Hz was not completely eliminated by synaptic blockers, the results suggest that the response consists of two components: a portion arising from direct activation of the ganglion cell and a portion that is synaptically mediated. The responses to 100 Hz (FIG. 3c) and to pulses (FIG. 3d) were affected very little by synaptic blockers suggesting the response to these waveforms arose predominantly from direct activation of the ganglion cell.

To further confirm that the spiking responses to LFSS resulted from modulation of synaptic input to the ganglion cell, a whole-cell patch clamp was used to record ganglion cell input currents. This allowed elimination of the possibility that the application of synaptic blockers simply reduced the level of tonic glutamate release from bipolar cells, thus decreasing the sensitivity of ganglion cells to electric stimulation. Voltage clamping at ECl and stimulating at 5 Hz gave a response that consisted of both the stimulus artifact and any inward (excitatory) currents (FIG. 4). Because the stimulus artifact is zero-mean, and the response shifted towards negative currents, stimulation at 5 Hz had elicited inward currents. This further supports the view that the spiking response of ganglion cells to LFSS results from activation of presynaptic neurons.

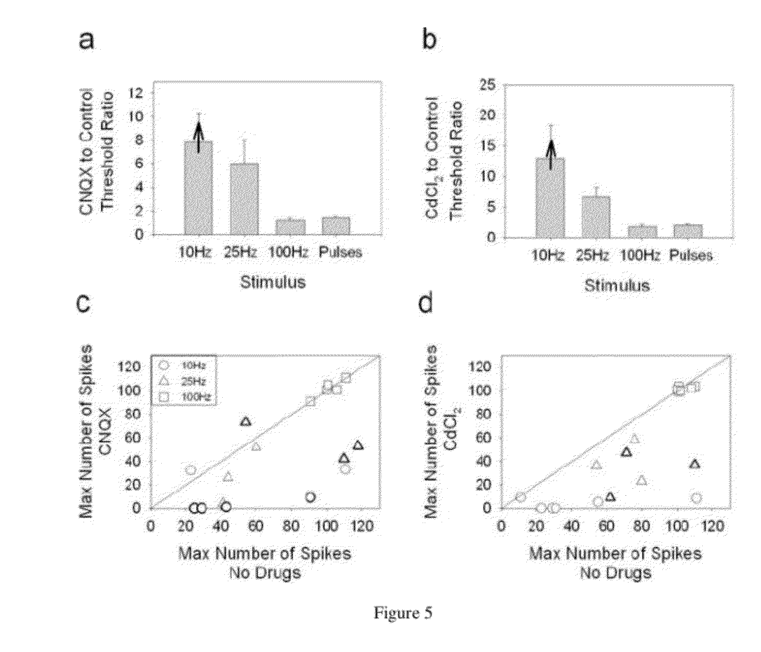

The effect of synaptic blockers was quantified in two ways. First, the response threshold in control conditions to the response threshold in CNQX or CdCl₂ were compared. The ratio of thresholds before and after the application of synaptic blockers for each stimulus waveform is shown in FIG. 5a-5b, indicating that responses to 10 and 25-Hz stimulation were more strongly affected by the blockers than responses to 100-Hz and pulsatile stimulation. These results are consistent with the view that the response to 10 and 25-Hz sinusoidal stimulation activate neurons presynaptic to the ganglion cell, while the response to 100-Hz sinusoids and 0.2-ms pulses are mediated by direct activation of the ganglion cell. Thresholds increased significantly in the presence of each blocker for stimulation at 10 Hz (p<0.001 for CdCl₂ and p<0.05 for CNQX) and 25 Hz (p<0.001 for CdCl₂ and p<0.02 for CNQX). The effect of the blockers was not significant for 100-Hz stimulation (p>0.07 for CdCl₂ and p>0.6 for CNQX), but was statistically significant for 0.2-ms pulses (p<0.001 for CdCl2 and p<0.01 for CNQX).

The second method used to quantify the level of synaptic input was to compare the maximum number of elicited spikes in control conditions versus the maximum number of spikes elicited with synaptic blockers (FIGS. 5c, 5d). The data for 10-Hz stimulation in either CNQX (FIG. 5c) or CdCl₂ (FIG. 5d) were largely clustered around the x-axis, again suggesting that synaptic input underlies most of this response. In contrast, the data from 100-Hz stimulation were largely clustered around the line of unity slope, confirming that synaptic input had little effect. The results for 25 Hz were mostly scattered between the line of unity slope and the x-axis, consistent with the response to 25 Hz arising from both presynaptic and direct activation.

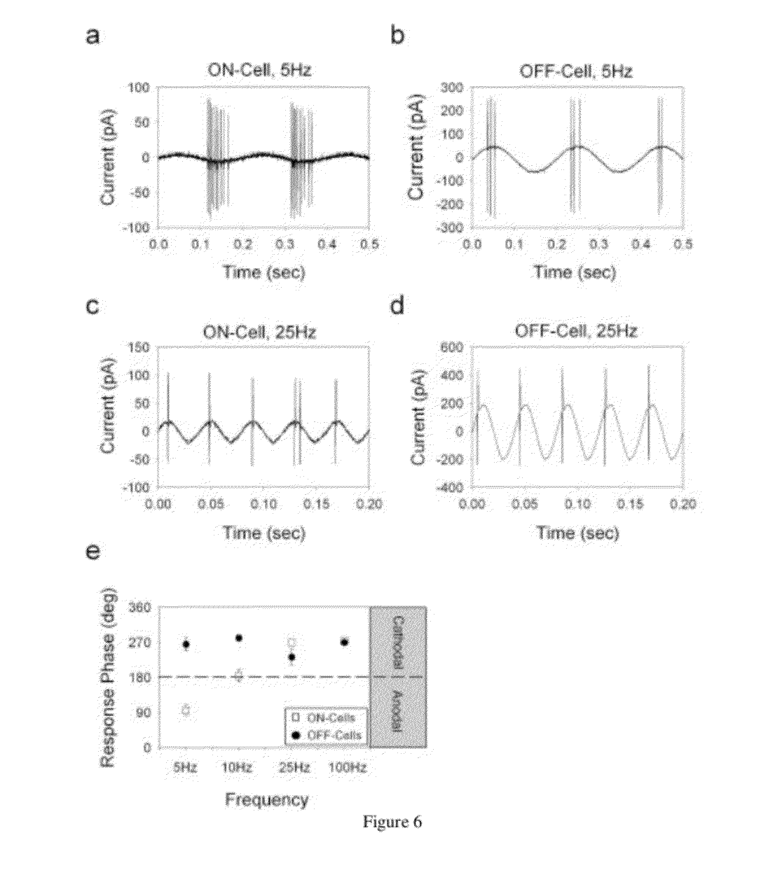

The present invention provides for the preferential activation of individual neuronal classes. Surprisingly, the class of presynaptic neurons activated by LFSS could be altered by changes in the stimulus frequency. In response to 5-Hz stimulation delivered near the soma, spikes occurred near the peak of the cathodal phase for OFF ganglion cells (n=6) (FIG. 6b) as expected (Ranck, 1975; Tehovnik et al., 96 J. Neurophysiol. 512 (2006)), but spikes occurred near the peak of the anodal phase for ON-type ganglion cells (FIG. 6a) (n=6). Because ON and OFF ganglion cells are thought to have similar intrinsic properties (O'Brien et al., 2002), the mechanism responsible for this ON and OFF difference could originate at a site presynaptic to ganglion cells, perhaps at the photoreceptor-to-bipolar cell synapse in the outer retina, where the ON and OFF pathways diverge. Thus, if the cathodal phase of the 5-Hz sinusoidal stimulus depolarizes photoreceptors, it would lead to a depolarization of OFF-bipolar cells and subsequent increased spiking in OFF ganglion cells. The same depolarization of photoreceptors would lead to hyperpolarization of ON bipolar cells because of sign inverting metabotropic glutamate receptors (mGluR) found in their dendrites, and a corresponding reduction in spiking for ON ganglion cells. Analogously, the anodal phase of 5-Hz stimulation would hyperpolarize photoreceptors which would decrease glutamatergic input to, and depolarize, ON bipolar cells, thereby causing increased spiking in ON ganglion cells.

Increasing the stimulus frequency to 25 Hz resulted in spikes that occurred exclusively near the cathodal peak for both ON and OFF cells (FIGS. 6c, 6d). This suggests that the mechanism of excitation at 25 Hz shifts to a location downstream of photoreceptors, most likely in bipolar cells since CNQX blocks much of this response. A summary of the average phase at which spiking occurs in ON and OFF cells for each stimulus frequency tested (FIG. 6e) shows that the ON and OFF phase differences occur for 5 Hz and 10 Hz, but for frequencies of 25 Hz and above the response phase remains cathodal for both cell types.

The frequency-dependent response properties of retinal neurons to electric stimulation are likely to be influenced by the properties of voltage-gated ion channels. It is well established that different types of ion channels are distributed heterogeneously across different classes of retinal neurons, as well as between different sub-regions of a given neuron. Because the kinetics by which different types of ion channels respond to changes in membrane voltage can also vary considerably, the possibility exists that differences in the frequency sensitivity observed experimentally may arise from differences in the distribution and/or kinetics of the ion channels inherent within the different classes of retinal neurons.

This possibility was explored using a computational model to examine the response of ion channels to different frequencies of sinusoidal stimulation. The channels tested were those thought to underlie the physiological responses observed: voltage gated sodium channels that underlie the spiking response in ganglion cells and are found in high densities in the proximal axon; and both L- and T-type calcium channels that have been shown to modulate synaptic release in bipolar cells and photoreceptors (only the L-type calcium channel has been identified in photoreceptors). These model ion channels were examined individually so that the frequency-dependent response properties of each could be isolated. The voltage across the channel was modulated and the resulting current was calculated according to equations that were based on previous studies. See Schutter & Bower, 71 J. Neurophysiol. 375 (1994); Benison et al., 210 J. Theor. Biol. 187 (2001).

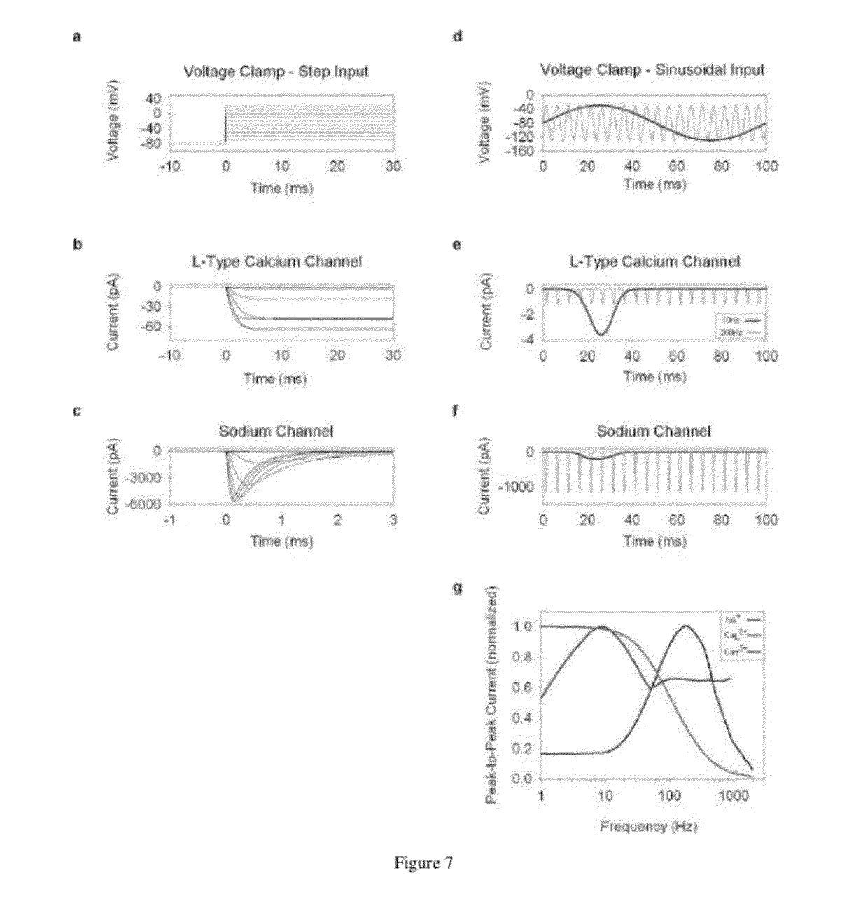

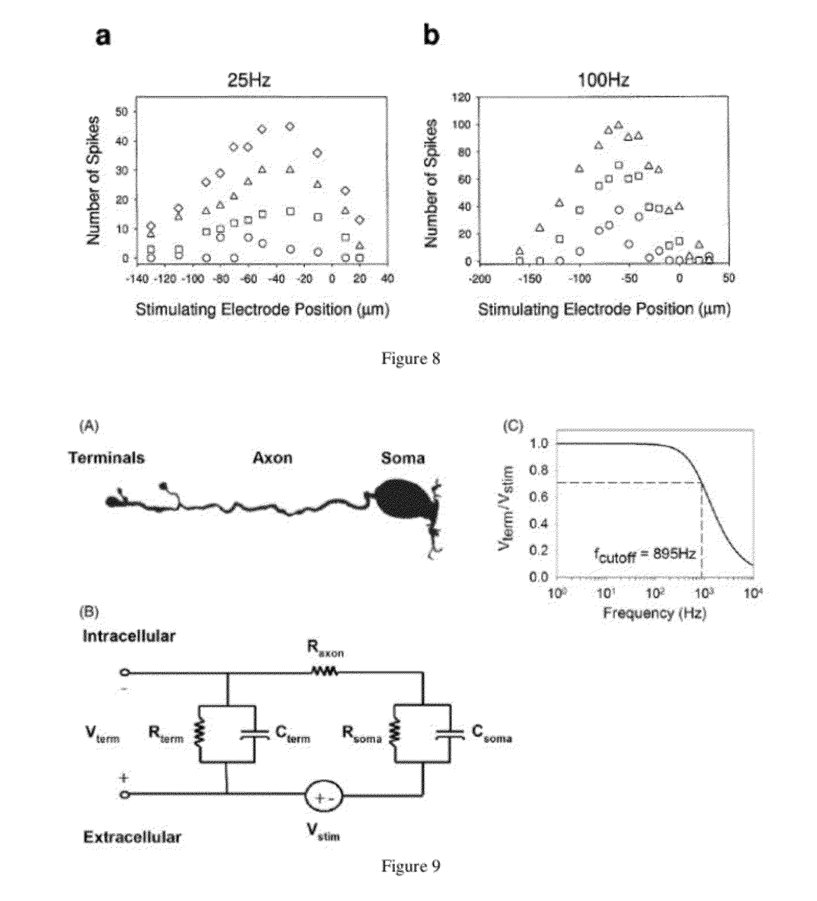

To test the kinetics and activation/inactivation properties of the individual ion channels within our model, steps of voltage were applied and the resulting current through each ion channel was calculated (FIG. 7a-c). Consistent with previous studies, the L-type calcium channel activated slowly with a time course of several milliseconds (FIG. 7b) (Protti & Llano, 18 J. Neurosci. 3715 (1998)), while the sodium channel activated rapidly (FIG. 7c), opening in less than a millisecond (Fohlmeister & Miller, 78 J. Neurophysiol. 1935 (1997)). The inactivating mechanism of the sodium channel also acts fairly quickly (˜1 ms); causing the sodium channel to close in response to sustained depolarization and reducing the sodium current back to baseline. Because the model L-type calcium channel does not inactivate, the L-type calcium current persists for the duration of the voltage step. Similar to the response of the sodium channel, the activation and inactivation mechanisms of the T-type channel combine to cause a transient increase in current in response to a step depolarization. Because the activation and inactivation kinetics of T-type channel are both slower than that of the sodium channel, however, the current increase starts after and persists longer than that of the sodium current.

To determine whether the response kinetics and activation/inactivation properties of the ion channel might contribute to the frequency-dependence observed experimentally, voltage was varied sinusoidally and the resulting sodium and calcium currents were calculated. Example response currents elicited by low (10 Hz) and high (200 Hz) frequencies are shown in FIG. 7d-7f. The response of the L-type calcium channel was significantly stronger in response to low frequency stimulation than to high frequency stimulation. The slow activation kinetics of the L-type channel was responsible for the weaker response at the high stimulus frequency. In contrast, the rapid activation kinetics associated with the sodium channel enabled the channel to open and close in response to the relatively rapid fluctuations in voltage associated with the high stimulus frequency. The weak response to the low frequency stimulus was due to the relatively fast inactivation mechanism of the sodium channel.

To determine the optimal range of stimulus frequencies for each model channel, the peak-to-peak current was calculated as a function of stimulus frequency (FIG. 7g). As suggested by the results at 10 Hz and 200 Hz (FIG. 7d-7f), the L-type calcium channel elicited strong responses at low frequencies but responded only weakly to higher stimulus frequencies. In contrast, the strongest response of the sodium channel was observed for a range of relatively high stimulus frequencies (centered around 200 Hz), consistent with its relatively fast activation kinetics. As with the step depolarization, the inactivation mechanism closes the sodium channel during slow depolarizations, thus suppressing the response to low frequency stimulation. Similar to the sodium channel, the T-type channel also exhibited a bandpass response. The relatively slow activation kinetics of the T-type channel resulted in an optimum frequency of ˜10 Hz, however, much lower than that of the sodium channel. The presence of an inactivation mechanism in the T-type channel limits the responsiveness at very low frequencies—similar to that of the sodium channel. The general shape of the frequency response was similar across a wide range of initial membrane voltages and stimulus amplitudes.

Interestingly, the model T-type channel maintained a moderate response level even at the highest frequency simulated (1000 Hz) (FIG. 7g). Although the fluctuations in voltage were too rapid to cause changes to the activation or inactivation state of the channel, the steady-state conductance in response to high frequency stimulation was nonzero (i.e., the activation and inactivation variables k and q were significantly different than zero). Therefore, current flowed through even for rapid fluctuations in voltage. The same was not true for sodium and L-type calcium channels, where the channels were closed in response to high frequency stimulation, preventing any current from flowing through the channel.

These computational results are highly consistent with the physiological data. For example, the ganglion cell responses that were mediated by activation of pre-synaptic neurons (FIG. 3) were strongest at low frequencies. This is consistent with the model results in which calcium channels, known to mediate synaptic release, responded optimally to low stimulus frequencies (FIG. 7g). Additional correspondence between the model and experimental results arise from comparison of the direct (non-synaptic) activation of ganglion cells (physiological) to the frequency-dependent characteristics of the model sodium channel. Previous physiologic data have suggested that the direct activation of ganglion cells is mediated by sodium channels, and the model predicted that sodium channels respond optimally to high stimulus frequencies. This is consistent with our experimental findings in which direct (non-synaptic) activation of ganglion cells is strongest for high frequency stimulation. The correspondence between the physiological and modeling results suggests that the activation and/or inactivation kinetics of voltage-gated ion channels is likely to contribute to the frequency-dependent response of neurons in response to electric stimulation. Therefore, customizing stimuli based on the frequency-dependent characteristics of voltage-gated ion channels endogenous to the target neuron may optimally activate the target neuron(s), and allow selective activation of individual classes of neurons or neuronal substructures.

Additionally, the ability of low frequency sinusoids to preferentially activate the indirect response may be advantageous in allowing existing inner retinal circuitry to be utilized, presumably resulting in spike trains that better resemble those that are present in the healthy retina. In addition, low frequency sinusoids avoid the activation of passing axons; this is thought to be critical for generating spatially focal percepts. The mechanisms underlying the preferential activation of the indirect response at low stimulus frequencies were unresolved. There are at least three major factors that influence the neuronal response to electric stimulation, each of which may contribute to the observed frequency-dependence. First, the membrane potential will be altered by the direct action of the electric stimulus on the targeted neuron; the magnitude and timing of any changes in membrane potential will depend on the passive electrical properties of the neuron (e.g., the resistance and capacitance of the cell membrane). Rattay, 45 IEEE Trans. Biomed. Engin. 766 (1998); Gerhardt et al., 18 IEEE Trans. Neural Sys. Rehab. Engin. 1 (2010). Second, changes in membrane potential will alter the flow of current through voltage-gated ion channels (McIntyre & Grill, 1998; Greenberg et al. 1999; Boinagrov et al., 104 J. Neurophysiol. 2236 (2010). The magnitude and timing of these currents will depend on the gating kinetics of the associated ion channel. Third, modulations in the level of excitatory and/or inhibitory input can occur if presynaptic neurons are also activated by stimulation. Fried et al., 2006; Margalit & Thoreson, 2006.

Therefore, the contribution of the passive membrane properties and voltage-gated calcium channels of bipolar cells to the preferential activation of the indirect response at low frequencies was explored. These two mechanisms are both intrinsic to the bipolar cell while the influence of presynaptic neurons were considered extrinsic effects and were excluded from this study. Previous work has raised the possibility that both intrinsic mechanisms described above could contribute to the preferential activation seen physiologically. For example, a recent modeling study found that the passive membrane properties of bipolar cells may act to low pass filter the applied stimulus (Gerhardt et al., 2010). This suggests that membrane potential is modulated more strongly at low stimulus frequencies. In addition, while several different types of voltage-gated ion channels have been identified in bipolar cells (Ma et al., 22 Vis. Neurosci. 119 (2005)), calcium entry into the axon terminals through L- and T-type calcium channels is known to underlie synaptic release. Tachibana et al., 707 J. Neurosci. 359 (1993); Pan et al., 32 Neuron 89 (2001). Because these channels have relatively slow opening kinetics (Pan, 83 J. Neurophysiol. 513 (2000); Pan et al., 2001), it is possible that calcium influx is stronger at low stimulus frequencies than high frequencies (even though the membrane potential of bipolar cells is modulated equally at both low and high stimulus frequencies).

To distinguish between these possibilities, a series of computational models that allowed assessment of the contribution of each factor in isolation. This revealed that the passive membrane properties of the bipolar cell did not influence the response for frequencies below several hundred hertz (cutoff frequency of 717 Hz for a ‘typical’ bipolar cell). In contrast, calcium channels responded maximally to relatively low stimulus frequencies (peak frequency of 5 to 25 Hz for T-type channels, and cutoff frequency of 65 to 500 Hz for L-type channels). Thus, these results suggest that the slow kinetics of calcium channels and not the passive membrane properties limit synaptic release in response to sinusoidal electric stimulation.

More specifically, in order to elucidate the mechanisms underlying this frequency-dependence, the contribution of passive electrical properties (i.e., no voltage-gated channels) of the bipolar cell membrane to the frequency response was examined using two approaches. First, the bipolar cell was represented by a two-compartment model. The simplicity of this model allowed the transfer function to be derived analytically using linear circuit analysis. Second, a morphologically realistic multi-compartment model was implemented in order to account for the complex morphological structure of a bipolar cell. Then, after the contribution of the passive electrical properties to the frequency response was evaluated, we examined the frequency response of L- and T-type calcium currents. These channels were first studied in isolation, and then inserted into the multi-compartment model.

The two-compartment model consisted of one compartment for the soma and one compartment for the terminal region (FIG. 9B). Each compartment contained a single resistor and capacitor in parallel. The two compartments were connected by a single resistor, representing current flow along the interior of the axon. Extracellular stimulation was simulated by placing a battery across the soma and terminal regions (Vstim). Because synaptic release results from depolarization of the synaptic terminals, we used the membrane potential in the terminals (Vterm) as a measure of bipolar cell activation in response to sinusoidal extracellular stimulation (Vₛₜᵢₘ). The advantage of the two-compartment model is that it allowed an analytical solution to be derived with basic circuit analysis. The circuit equations in the frequency domain were derived because to examine the response to sinusoidal stimulation. Oppenheim et al., 2 J. Neural Engin. 5105 (1996). The impedances of the soma, axon, and terminals are represented in the Laplace domain as follows:

Zₛₒₘₐ=Rₛₒₘₐ/(1+sRₛₒₘₐCₛₒₘₐ) (1)

Zₐₓₒₙ=Rₐₓₒₙ (2)

Zₜₑᵣₘ=Rₜₑᵣₘ/(1+sRₜₑᵣₘCₜₑᵣₘ) (3)

where s is complex frequency. Vₜₑᵣₘ was solved for using a voltage divider:

Vₜₑᵣₘ=Vₛₜᵢₘ(Zₜₑᵣₘ(ZₜₑᵣₘZₛₒₘₐZₐₓₒₙ)) (4)

Nominal values for resistance and capacitance for each compartment were derived from previous work. Oltedal et al., 587 J. Physiol. 829 (2009). With these parameter values, the transfer function (Vₜₑᵣₘ/Vₛₜᵢₘ) was computed for frequencies ranging from 1 Hz to 104 Hz (FIG. 9C). The sensitivity of the response (Vₜₑᵣₘ) to the applied stimulus (Vₛₜᵢₘ) was nearly constant up to several hundred hertz and then declined steadily for increasing stimulus frequencies. Therefore, the relationship between the applied stimulus and the membrane potential in the terminals exhibits lowpass filtering characteristics. The cutoff frequency was defined as the frequency at which the response is reduced by 3 dB from maximum; the cutoff frequency for the nominal parameter values was 895 Hz. This suggests that the passive membrane properties of the cell have little effect on the response out to relatively high frequencies.

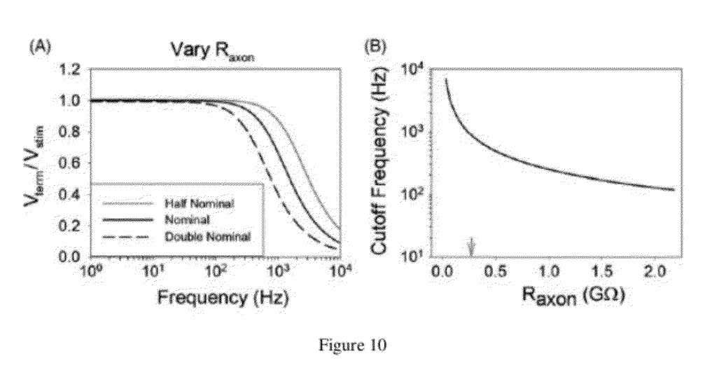

There are at least ten different types of bipolar cells whose size and morphology vary considerably (Euler & Wassle, 361 J. Comp. Neurol. 461 (1995); Boycott & Wassle, 40 Invest. Ophthalmol. Vis. Sci. 1313 (1999); Wu et al., 20 J. Neurosci. 4462 (2000). To determine how these anatomical differences might influence the frequency response, each model parameter was systematically varied and the effect on the transfer function explored. For example, the transfer function was computed for an axonal resistance (Raxon) value of nominal, one-half nominal, and twice nominal (FIG. 10A). As Raxon was increased, the cutoff frequency decreased. Interestingly, variations in Raxon had a negligible influence on the response to low-to-moderate frequencies (<102 Hz), suggesting that variations in intra-axonal resistance only effect the cutoff frequency.

To determine the range of cutoff frequencies arising from changes in axonal resistance (Raxon), the cutoff frequency was computed for values of Raxon ranging from ⅛ to 8× its nominal value (FIG. 10B). This range of axonal resistance corresponds to a factor of 2 change in both axonal length and axonal diameter. For example, if the length of the axon is doubled and the diameter is halved, then the resistance of the intra-axonal current flow will increase by a factor of 8 (the change in cross-sectional area increased resistance by a factor of 4 and the change in length increased the resistance by a factor of 2). This range of parameters likely spans the range of bipolar cell morphology seen across bipolar cell types. The cutoff frequency was found to decrease for increasing axonal resistance (i.e., longer, thinner axons), but remained relatively high (>117 Hz) even for the largest value of Raxon tested. In contrast, the cutoff frequency increased dramatically for decreasing axonal resistance (i.e., shorter, wider axons). Given that the cutoff frequency tends to plateau for high values of Raxon (FIG. 10B), it is likely that the cutoff frequency will remain relatively high for all anatomically realistic variations in axonal resistance.

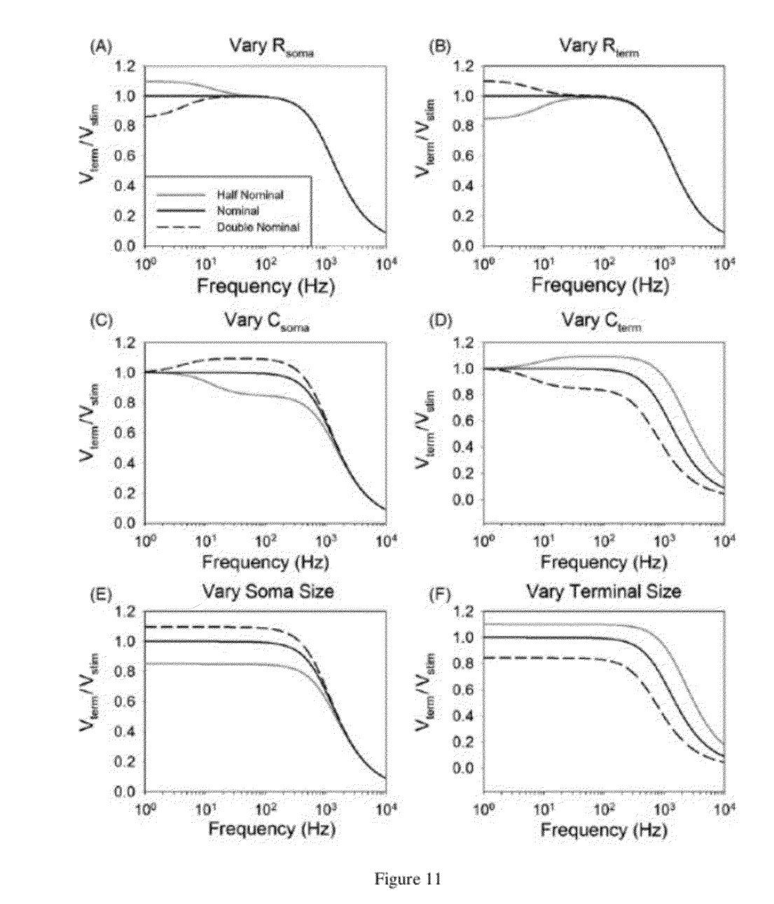

Changes in the size of the soma or terminals will change both the resistance and capacitance of each compartment. Therefore, the resistance and capacitance were varied independently in order to determine the individual contributions of each to the transfer function. Interestingly, varying the resistance of the soma (Rₛₒₘₐ) or terminals (Rₜₑᵣₘ) affected only the low frequency portion (<10 Hz) of the transfer function, leaving the cutoff frequency unchanged (FIG. 11A-11B). Changing the somatic or terminal resistance had opposing affects; increasing the somatic resistance caused a reduction in gain at low frequencies, while increasing the terminal resistance caused an increase in gain at low frequencies. The effect on gain was modest; in each case, changing the resistance by a factor of two caused a change in gain ˜15.2% for stimulation at 1 Hz.

Changes in the capacitance of the somatic or terminal regions also altered the transfer function. For changes in the somatic capacitance, the effect was confined to the range of 10 Hz to 103 Hz, while changes to the terminal capacitance affected all frequencies>10 Hz (FIG. 11C-11D). In both cases, there was relatively little effect for frequencies<10 Hz. Changes in the capacitance of the soma or terminals had opposing effects; increasing the capacitance of the soma caused an increase in gain while increasing the capacitance of the terminals had a decrease in gain.

Understanding how the resistance and capacitance influences the transfer function allowed the effects of anatomical changes (e.g., soma size) to be more easily understood. The effect of varying soma or terminal size was performed by adjusting both the resistance and capacitance of a given compartment. For example, doubling the size of the soma was performed by increasing the capacitance by a factor of 2 and simultaneously decreasing the membrane resistance by a factor of 2. Increasing soma size produced an increase in gain at frequencies of <103 Hz, leaving higher frequencies relatively unaffected (FIG. 11E). Conversely, increasing the size of the terminals caused a reduction in gain across all frequencies (FIG. 11F).

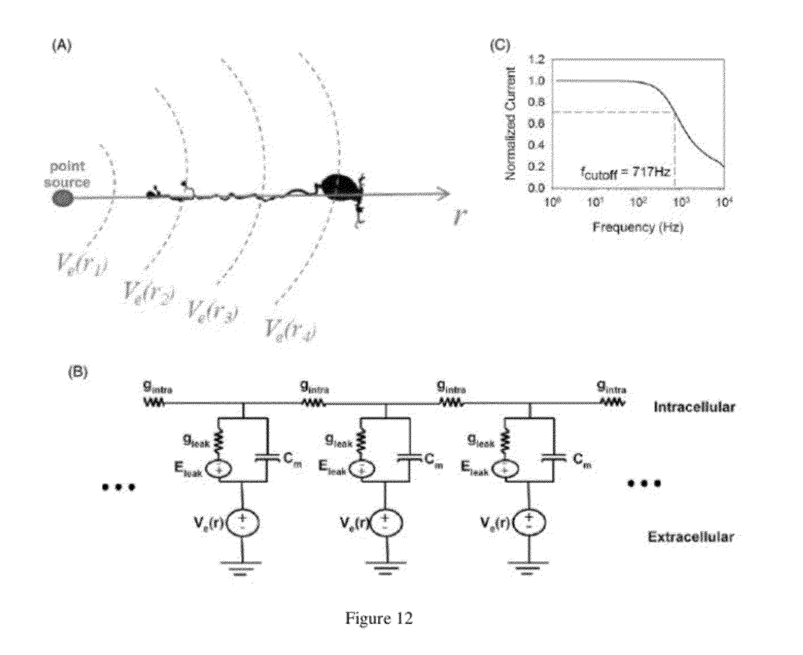

Although the two-compartment model has the advantage of allowing an analytical solution to be derived, it does not account for the complex morphological structure of a bipolar cell. Therefore, the response properties of a morphologically realistic, multi-compartment bipolar cell were examined. Oltedal et al., 2009; (FIG. 12A-12B). Each compartment was defined by a membrane conductance (gleak) and capacitance (Cm), as well as by resistance to current flow along the interior of the cell (gintra). The conductance gintra was computed as a function of intra-cellular resistivity (ρi). The membrane potential at the terminals (Vterm) was measured in response to sinusoidal stimulation delivered from an extracellular electrode (Ve). As with the two-compartment model, the multi-compartment model did not contain voltage-gated channels initially so that the contribution of the passive electrical properties of the membrane to the frequency response could be studied in isolation.

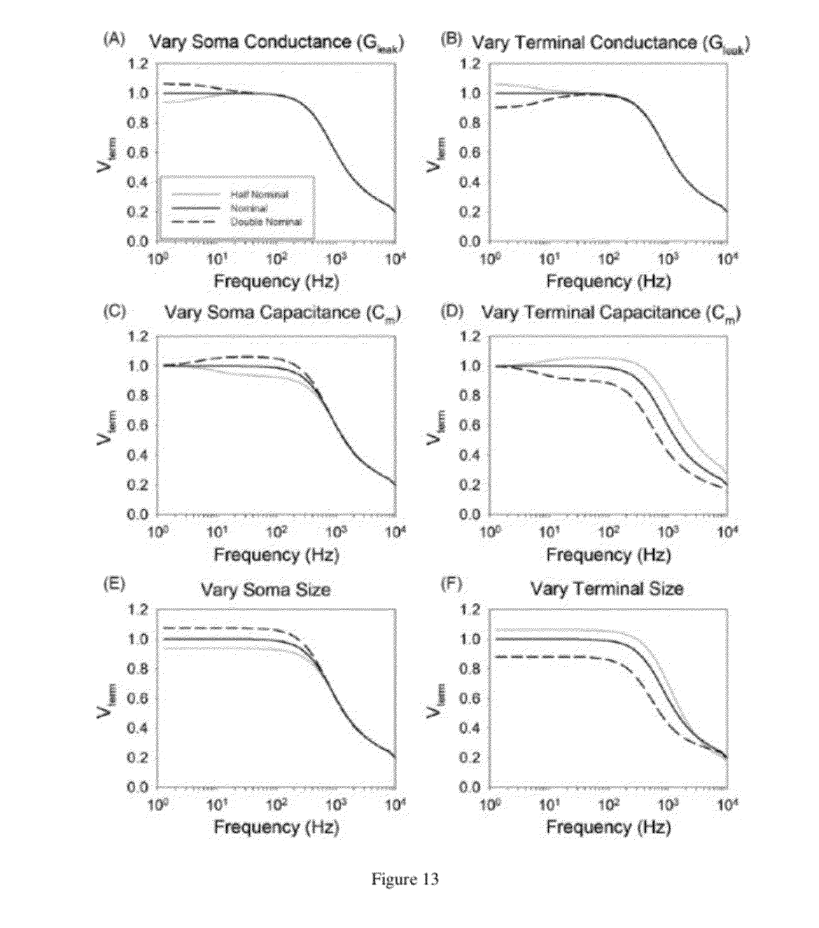

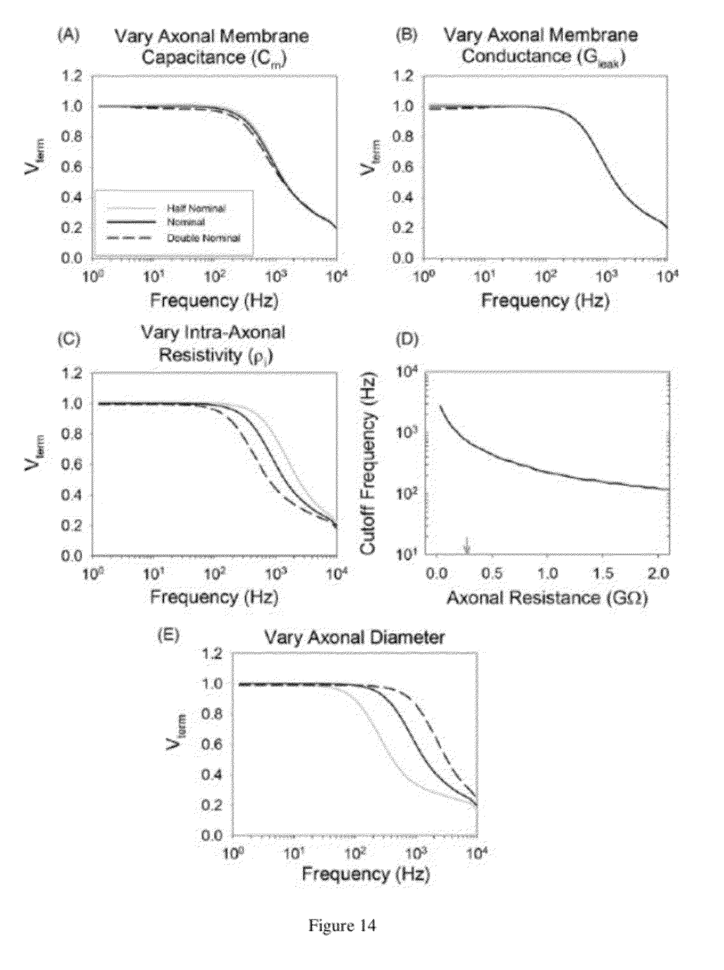

In general, the responses of the morphologically realistic bipolar cell model were similar to those obtained for the two-compartment model. The frequency response was lowpass with a cutoff frequency of 717 Hz for the nominal parameter values (FIG. 12B). Once again, the curve obtained using nominal parameter values is re-plotted in FIGS. 13 to 15 to facilitate comparison (labeled as Nominal'). As with the two-compartment model, varying the somatic or terminal membrane conductance only influenced the response to low frequencies (<10 Hz) (FIG. 13A-13B), while varying the capacitance affected the response to frequencies>10 Hz (FIG. 13C-13D). Likewise, varying the size of the soma or terminals in the multi-compartment model produced a nearly identical affect as that seen in the two-compartment model (FIG. 13E-13F vs. FIG. 11E-11F). Unlike the two-compartment model, the axon of the multi-compartment model has a membrane with an associated conductance and capacitance. Variations in the conductance or capacitance of the axonal membrane had little effect on the frequency response (FIG. 14A-14B).

Examining the influence of intra-axonal resistance on the frequency response in the multi-compartment model was more complex than the corresponding analysis in the two-compartment model. This is because the intra-axonal resistance for the two-compartment model is defined by a single parameter, Raxon, while the intra-axonal resistance in the multi-compartment model was a function of axonal diameter, axonal length, and the resistivity of the intracellular medium (ρi). The influence of each of these parameters on the frequency response was examined. Changes to the intra-axonal resistivity influenced the cutoff frequency, but had little effect on gain (FIG. 14C). The cutoff frequency was measured as a function of intra-axonal resistivity (FIG. 14D), and in order to allow a direct comparison to the two-compartment model to be made, the results are plotted as a function of total intra-axonal resistance (not resistivity). The dependence of cutoff frequency on intra-axonal resistance was nearly identical for the two models (compare FIG. 14D vs. FIG. 10B).

Next, the effect of varying axonal diameter on the frequency response was examined. Changes to axon diameter will affect the total membrane conductance and capacitance, as well as the resistance to intra-axonal current flow. Because changes to the axonal membrane conductance and capacitance had little effect on the frequency response (FIG. 14A-14B), it was expected that changing the axonal diameter would produce results that were similar to those when the intra-axonal resistance was changed. This was found to be the case, as varying axonal diameter influenced the cutoff frequency, but had little effect on the response to low stimulus frequencies (FIG. 14E). Smaller axonal diameters resulted in increased resistance to intra-axonal current flow, producing a lower cutoff frequency.

How changes to axonal length effected the frequency response was also explored. In the model, changes to axonal length altered the distance between the axon terminals and the stimulating electrode. Moving the terminals closer to the stimulating electrode, however, would increase the sensitivity of the cell to the applied stimulus. Therefore, it was necessary to decouple the effects due to changing axonal length from those due to changing the position of the stimulating electrode. This was performed in a two-step process:

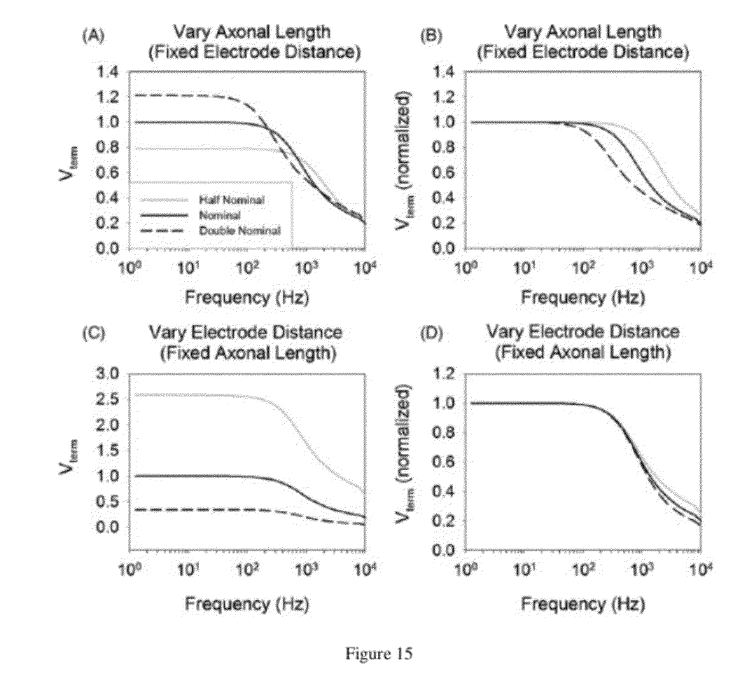

First, the frequency response for variations in axonal length with the electrode fixed to a set distance (40 μm) from the terminals was measured (FIG. 15A). Under these conditions, increasing the length of the axon caused an increase in gain, as well as a decrease in cutoff frequency. Normalizing and overlaying the curves obtained for each axonal length revealed that longer axons are associated with lower cutoff frequencies (FIG. 15B). This finding is consistent with previous simulations that showed that increases in intra-axonal resistance resulted in a decrease in cutoff frequency (FIG. 14D). The increased gain for longer axons likely results from the larger spatial gradient in extracellular potential that exists across longer axons as compared to shorter axons.

Second, axonal length was held constant and measured the frequency response as the distance between the stimulating electrode and the terminals was varied (FIG. 15C). As expected, the sensitivity to stimulation was greatly increased when the distance between the stimulating electrode and the terminals was reduced. Normalizing and overlaying these responses reveals that electrode distance does not significantly affect cutoff frequency (FIG. 15D). Taken together, the results from FIG. 15 indicate that longer axons are associated with a decrease in cutoff frequency and an increase in sensitivity to low stimulus frequencies. Conversely, changes to the distance between the bipolar cell and the stimulating electrode effects the response to all frequencies uniformly (i.e., does not alter cutoff frequency).

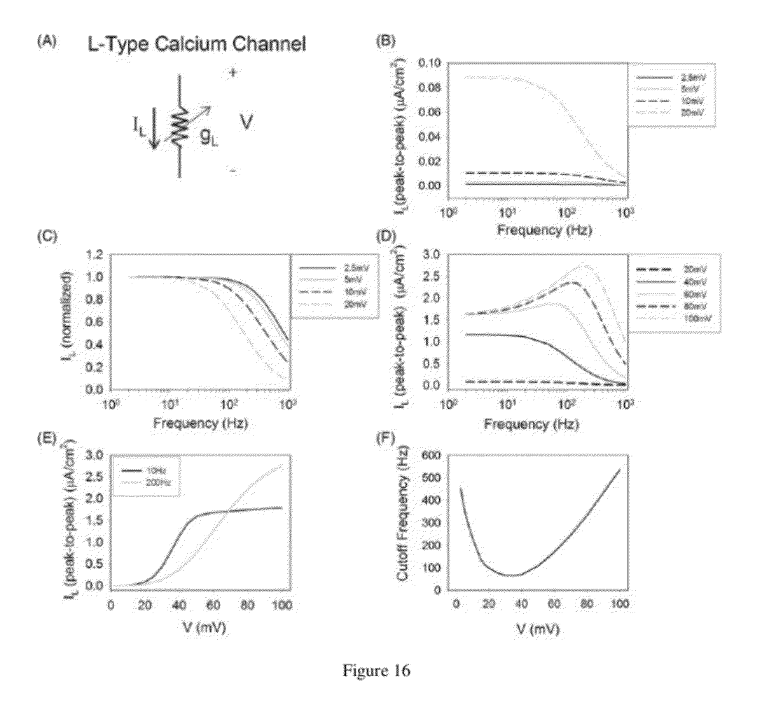

Synaptic release from bipolar cells is mediated by calcium entry to the terminals via L- and/or T-type calcium channels (Tachibana, 1993; Pan et al., 2001). These channels are voltage dependent, and equations that describe the relationship between membrane potential and the probability of opening (and thus calcium conductance) have been derived for L- and T-type channels in other types of neurons and were adopted for use in our model. De Schutter & Bower, 71 J. Neurophysiol. 375 (1994); Benison et al., 2001. The frequency-dependent response properties of these channels were examined by modulating voltage (V) sinusoidally across a range of frequencies and measuring the resulting current (IL and IT) (FIG. 16A). Unlike the linear passive model, the gating equations are nonlinear, and therefore the frequency response may depend on amplitude of the applied voltage.

The voltage was oscillated around a mean of −50 mV; this value was chosen to approximate the resting potential of bipolar cells. Ma et al., 2005. In response to light, the fluctuations in bipolar cell membrane potential is thought to saturate near 15-25 mV. Nelson & Kolb, 23 Vis Res. 1183 (1983); Euler & Masland, 83 J. Neurophysiol. 1817 (2000). In response to electric stimulation, it is possible that much larger fluctuations in membrane potential could occur. Therefore, the behaviors of calcium channels for two ranges of voltage fluctuations were examined: a physiologically realistic range (deviations of 2.5-20 mV from baseline) and a larger range that could potentially be induced by extracellular electric stimulation (deviations of 40-100 mV from baseline).

In response to voltage fluctuations of ≦20 mV, L-type calcium channels exhibited lowpass filtering characteristics, yielding larger currents at low frequencies than at high frequencies (FIG. 16B). Normalizing and overlaying these response curves revealed that the cutoff frequency decreased slightly as stimulus amplitude increased (FIG. 16C). Interestingly, once the range of voltage fluctuations exceeded ˜40 mV, the shape of the frequency response changed from lowpass to bandpass (FIG. 16D). For voltage fluctuations>40 mV, the response at low frequencies became saturated, while the response to moderate to high frequencies continued to increase. This can be seen more clearly by plotting the peak current as a function of stimulus voltage for two different frequencies (10 Hz and 200 Hz) (FIG. 16E). Note that the maximal response currents achieved at a frequency of 200 Hz is nearly twice the maximal response at 10 Hz.

The cutoff frequency varied significantly as a function of stimulus voltage for L-type channels, exhibiting a parabolic shape with a minimum cutoff frequency of 65 Hz at ˜34 mV (FIG. 16F). Note that in order to compare the cutoff frequencies for the lowpass and bandpass frequency responses, the cutoff frequency was always defined as the highest frequency at which the response was reduced by 3 dB (i.e., not defined as the peak of the frequency response). Taken together, these data suggest there are two separate modes of behavior for L-type calcium channels. For moderate fluctuations in voltage, the frequency response is lowpass, and the cutoff frequency decreases as voltage increases. For higher fluctuations in voltage, the frequency response becomes bandpass and the cutoff frequency increases with voltage.

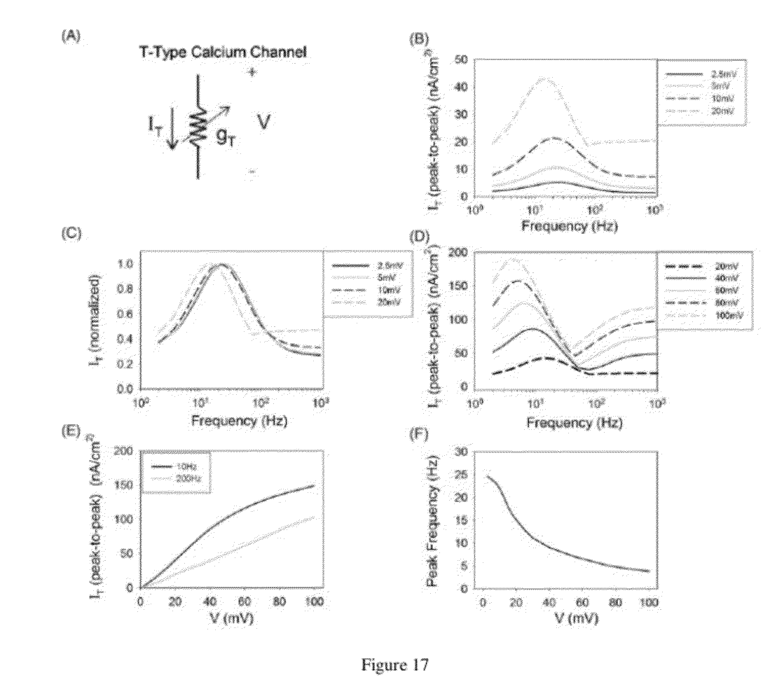

As with the L-type channel, the frequency response of T-type channels was obtained by varying the voltage (V) sinusoidally and measuring the resulting current (IT) (FIG. 17A). The frequency response of the T-type channels exhibited bandpass characteristics with a pronounced peak in sensitivity near 10 Hz (FIG. 17B). Normalizing and overlaying these response curves revealed that the general shape of the frequency response was maintained as voltage level was increased (FIG. 17C), although the peak shifted to slightly lower frequencies for higher voltage fluctuations (FIG. 17D). Currents through the T-type channel increased approximately linearly over the range of membrane potential levels tested for 200 Hz, whereas for 10 Hz stimulation there was slight response compression for voltage fluctuations>40 mV (FIG. 17E). The frequency at which the response was maximal (i.e. peak frequency) varied with stimulus voltage, but always remained below 25 Hz (FIG. 17F).

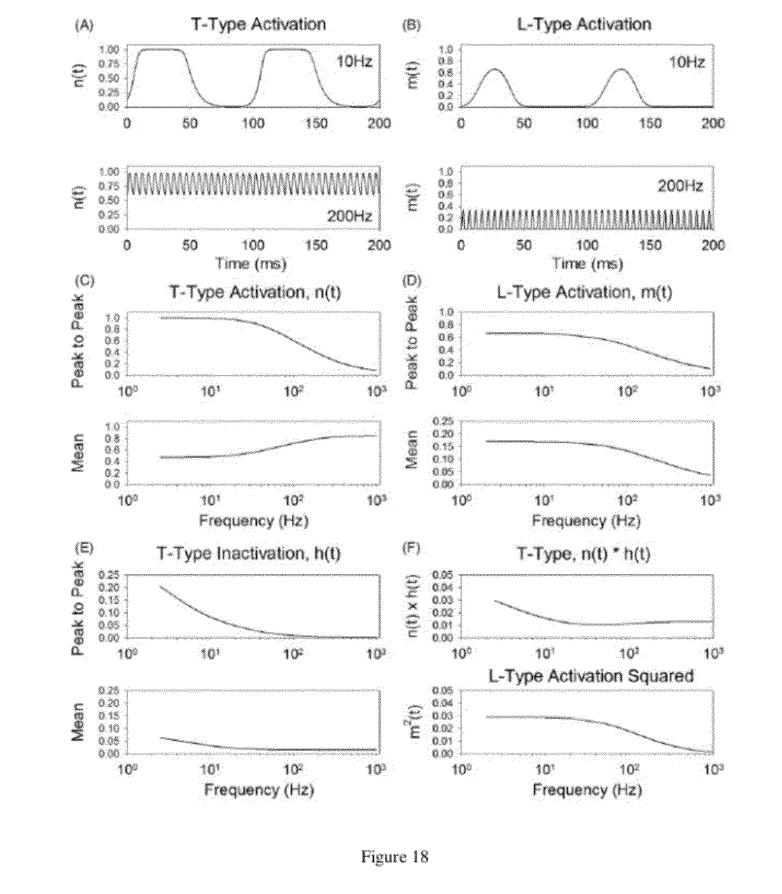

Surprisingly, current continued to flow through T-type channels even for high stimulus frequencies (FIG. 17B-17D). To explore why this occurred for T-type channels but not for L-type channels, the activation variables of L- and T-type channels, as well as the inactivation variable of the T-type channel, across a range of stimulus frequencies were examined (FIG. 18). The peak-to-peak value of n(t) (the T-type activation variable) decreased as stimulus frequency was increased from 10 Hz to 200 Hz, but the mean value of n(t) was larger at 200 Hz than at 10 Hz (FIG. 18A). This was not the case for the L-type activation variable, m(t), where increasing the frequency from 10 Hz to 200 Hz caused a reduction in both the peak-to-peak and mean value of m(t) (FIG. 18B). This point was further illustrated by measuring peak-to-peak and mean values of the activation and inactivation variables for frequencies up to 1 kHz (FIG. 18C-18E). Notice that the mean value of the T-type activation variable increased and plateaued at a nonzero value for high stimulus frequencies (FIG. 18C, bottom). For the stimulations in FIG. 18, the stimulus amplitude used was 40 mV, but similar behavior was observed for stimulus amplitudes ranging from 2.5-100 mV.

Because T-type channels contain both activation and inactivation gating parameters, it is necessary for both to be non-zero in order for current to flow through the channel at high stimulus frequencies. As FIG. 18C indicates, the activation variable, n(t), is much greater than zero (˜0.85) for high stimulus frequencies. Although the mean inactivation variable, h(t), decreases for increasing stimulus frequency (FIG. 18E), it plateaus at a nonzero value (˜0.015). Therefore, the steady-state conductance is nonzero for high stimulus frequencies. This is further illustrated by comparing the scaling factors used to compute conductance for each channel: for T-type channels this is the product of the activation and inactivation variables (n(t)*h(t)), and for L-type channels this is the square of the activation variable (m2(t)) (FIG. 18F). Notice that for T-type channels, this scaling factor plateaus for increasing stimulus frequency, while for L-type channels the scaling factor continues to decrease for increasing frequency. Thus, the results suggest that T-type channels allow calcium current to flow for very rapid fluctuations in voltage, even though the channels themselves cannot open and close at such high rates. This is not true for L-type channels since the activation variable continued to decrease for increasing stimulus frequency.

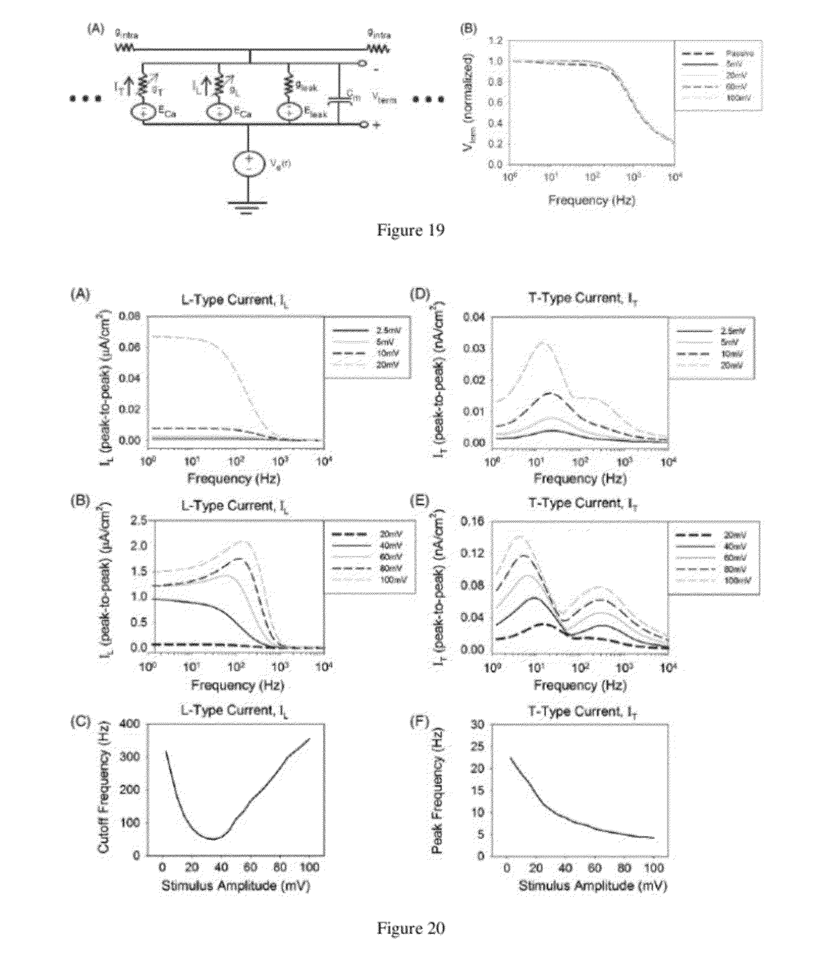

One of the original goals of this study was to understand why synaptic release from bipolar cells was elicited in response to extracellular sinusoidal stimulation at low frequencies (≦25 Hz), but not for high frequencies (100 Hz) (described herein; Freeman et al., 2010). The results suggest that the lack of synaptic release of bipolar cells in response to 100 Hz stimulation was not likely to be the result of passive filtering by the bipolar cell membrane. In addition, the simulations with calcium channels suggest calcium currents will be largest for low stimulus frequencies (tens of hertz). In order to make a direct comparison between the effects of calcium channel dynamics to those of passive membrane filtering, we inserted L- and T-type calcium channels into the terminal region of the multi-compartment model (FIG. 19A). Simulations were performed with both channel types inserted simultaneously, but the results did not differ if the L- and T-type channels were inserted independently.

The total membrane conductance for L- and T-type calcium channels in bipolar cells is not known, and therefore was set equal to the leak conductance (gLmax=gTmax=gleak). With these conductance levels, the frequency response of Vterm did not change appreciably when the channels were added (FIG. 19B). This indicates that for these values of calcium conductance, the presence of the calcium channels did not affect the relationship between the applied stimulus and Vterm. Although fluctuations in membrane potential may differ in the soma versus terminals, synaptic release takes place only in the terminals, and therefore we only inserted calcium channels into the terminals region. Because the presence of the calcium channels does not affect the response of the membrane potential to stimulation, the presence of calcium channels in other sections of the bipolar cell (e.g., the soma) will not influence the variable of interest—the current through the calcium channels in the terminals.

The current through L- and T-type calcium channels was measured in order to infer the level of synaptic release in response to extracellular sinusoidal stimulation. Stimulus amplitudes were adjusted to produce modulations in membrane potential (Vterm) in the range of 2.5-100 mV and the resulting L-type current (IL) and T-type current (IT) were measured (FIG. 20A-20B). Considering the L-type channels first, the shape of the frequency response went from lowpass to bandpass as stimulus amplitude increased. The cutoff frequency of the L-type current in the multi-compartment model was less than the cutoff frequency of the passive multi-compartment model (717 Hz for nominal parameters) for all stimulus amplitudes tested (FIG. 20C). Furthermore, the shape of the frequency response was identical to that obtained for the L-type calcium channel alone (FIGS. 20C vs. 16F). This suggests that the L-type channel mediated synaptic release from bipolar cells in response to electric stimulation is limited by the dynamics of L-type calcium channels and not by the passive properties of the membrane.

The frequency response for current through T-type calcium channels (IT) in the multi-compartment model was found to be bandpass (FIG. 20D-20E). The frequency yielding the largest response (i.e., the peak frequency) decreased for increasing stimulus amplitude, similar to the results obtained from T-type channels studied in isolation (FIG. 20F, compare to FIG. 17F). As with the L-type channels, the peak frequency of the T-type channel current was significantly less than the cutoff frequency of the passive membrane model for all stimulus amplitudes (maximum peak frequency=22.4 Hz). Also, for stimulus frequencies up to ˜200 Hz, the shape of the frequency response was similar to that obtained when studying the T-type channel in isolation (FIG. 20E, compare to FIG. 17D). This suggests that for stimulus frequencies of <200 Hz, T-type channel mediated synaptic release is largely determined by the dynamics of T-type channels, and not by passive membrane properties. At higher stimulus frequencies (>200 Hz), however, the current through T-type channels in the multi-compartment model decreased steadily for increasing frequencies (FIG. 20E), while the current through T-type channels studied in isolation reached a plateau (FIG. 17D). This reduction in current at high stimulus frequencies is due to passive filtering of the membrane, preventing the membrane potential (Vₜₑᵣₘ) from being modulated in response to rapid fluctuations of the stimulus (Vₑ). Therefore, the passive membrane properties may influence synaptic release for relatively high stimulus frequencies, while the response at frequencies of <200 Hz is largely determined by T-type channel dynamics.

Electric stimulation with sinusoidal waveforms provides a level of control over neuronal activation that has not been possible with more conventional pulsatile stimulation. LFSS avoids the activation of axons, while still eliciting robust responses in the target neuron. In addition, the specific class of neuron being activated depends on the frequency of sinusoidal stimulation: photoreceptors are activated at 5 Hz, bipolar cells at 10-25 Hz, and ganglion cells at 100 Hz. The ability to target specific classes of neurons has important implications for the retinal prosthetic as well as for a wide range of other neural prostheses.

One of the principal features of the present invention is that LFSS is much more effective than short-duration pulses at avoiding the activation of passing axons. Previous physiological studies found that for short-duration pulses, the threshold for activation of the distal axon was only two times greater than the threshold for activation for the soma region (Jensen et al., 2003). This is consistent with the present results, which found that the threshold ratio with short-duration pulses was ˜3 (FIG. 2). The slight difference between these findings and the previous study was likely due to the difference in stimulation parameters (0.2-ms vs. 0.1-ms pulses, 10-kΩ vs. 1-MΩ impedance of the stimulating electrode). The threshold ratios were significantly higher with LFSS: at 25 Hz the threshold ratio was >7 and for 10 Hz the ratio was >10. The ratios for LFSS are lower bounds since we could not elicit responses from the distal axon, even at the highest stimulus amplitudes that could be delivered safely. The higher ratios associated with LFSS suggest that it is a significant improvement for avoiding the activation of passing axons.

The ability to avoid the activation of passing axons in retinal prostheses will reduce the spatial spread of activation, potentially improving the control over the spatial pattern of the elicited percept. For example, in human trials, blind patients often report a percept that is oval in shape, and this is potentially due to incidental activation of passing axons. Horsager et al., 51 IOVS 1223 (2010). There are also other factors that influence the spatial pattern of elicited activity. Previous work has shown that increased stimulus amplitude for pulsatile stimuli activates cells further from the stimulating electrode, thus spreading the area of elicited activity (Jensen et al., 2003).

It is unlikely that variations in pulse rate would have a significant effect on the results. The responses to short-duration pulses arise predominantly from direct activation of the ganglion cell and not activation of presynaptic neurons (FIG. 3). Previous work has shown that the ability to elicit spikes through direct activation delivered near the soma varies little for pulse rates up to 100 Hz. (Sekirnjak et al., 28 J. Neurosci. 4446 (2006). Therefore, changes in pulse rate are not expected to have a significant effect on the relative threshold of the distal axon versus the soma region.

Another principal feature of the present invention is that changes to the frequency of sinusoidal stimulation altered the class of retinal neuron that was activated. This was inferred by observing the frequency-dependent change in the phase during which the responses were elicited. For example, OFF-ganglion cells tended to respond during the cathodal phase of the stimulus for both 5-Hz and 25-Hz stimulation. ON-ganglion cells, however, responded during the cathodal phase for 25-Hz stimulation, but responded during the anodal phase for 5-Hz stimulation (FIG. 6). Given that the traditional view of electric stimulation is that neurons are depolarized in response to cathodal stimulation, it was surprising that ON-ganglion cells elicited a response during the anodal phase. One explanation for the response differences between ON and OFF-cells is that photoreceptors are activated by 5-Hz stimulation; because the photoreceptor output is inverted at the ON-bipolar synapse (but not the OFF-bipolar synapse), depolarization of photoreceptors (during the cathodal phase) would elicit spiking in OFF-ganglion cells while hyperpolarization of photoreceptors (during the anodal phase) would elicit spiking in ON-ganglion cells. For 10-Hz stimulation, the spikes elicited in ON-ganglion cells occurred during the transition between the anodal and cathodal phase. The phase shift that occurs as the stimulus frequency increases from 5 Hz to 25 Hz suggests that the neural class activated shifts from photoreceptors to bipolar cells.

The possibility may exist that the ON/OFF phase difference for 5-Hz stimulation arises from the activation of horizontal cells and not photoreceptors. This is unlikely because the anticipated response polarity from horizontal cell activation is inconsistent with the data. For example, if the cathodal phase of the stimulus depolarizes horizontal cells, photoreceptors would be inhibited and there would be a reduction in glutamate release on to the bipolar cell dendrites. Because ON-bipolar cells depolarize in response to reduced glutamate input, ON-ganglion cells should exhibit increased spiking during the cathodal phase. This is inconsistent, however, with the observed data (FIG. 6), suggesting that the response at 5 Hz is most likely the result of photoreceptor activation.

In addition to activating photoreceptors and bipolar cells with stimulation at 5 Hz and 25 Hz, respectively, the present data suggest that ganglion cells can also be directly activated by increasing the stimulus frequency. The response of ganglion cells to 100-Hz stimulation was not significantly affected by the application of synaptic blockers (FIG. 5), consistent with the response arising primarily from direct excitation of the ganglion cell. Thus, the present results suggest that different classes of retinal neurons can be targeted with the appropriate tuning of stimulus frequency; photoreceptors at 5 Hz, bipolar cells at 25 Hz, and ganglion cells at 100 Hz. Although the ability to target photoreceptors is of limited use for retinal prostheses since these cells have degenerated, the ability to preferentially target specific classes of neurons has important implications. For example, in the retinal prosthetic, the ability to activate bipolar cells (e.g., at frequencies of 10-25 Hz) may be advantageous if it allows the inner retinal circuitry to be utilized and results in neural activity in ganglion cells that more closely resembles physiological signaling patterns.

The synaptically mediated response of ganglion cells to stimulation at 5-25 Hz was greatly reduced following application of CNQX. The additional application of cadmium was necessary to completely abolish the response, however. There are several possible sources for this CNQX-insensitive response component (difference between the traces in FIG. 3a), including acetylcholine from activation of starburst amacrine cells (Famiglietti, 261 Brain Res. 138 (1983)), glutamatergic activity mediated by NMDA receptors (Kalloniatis et al., 21 Vis. Neurosci 587 (2004)), or reduced inhibitory input to ganglion cells mediated via activation of a serial inhibitory pathway (Roska et al., 18 J. Neurosci. 3451 (1998)). It is also possible the presence of cadmium reduced the response by blocking voltage-gated calcium channels that are intrinsic to the ganglion cell. Benison et al., 2001. It is unlikely that these channels play a major role because direct activation of the ganglion cell is thought to be largely mediated by the dense band of sodium channels in the initial segment (Fried et al., 2009). Although the present data do not allow unequivocal determination of the origin of the CNQX-insensitive response, because most of the synaptic response was eliminated in CNQX, it is likely that increased bipolar cell output is the primary source of the synaptic response.

Although HFSS was effective at exciting the ganglion cell directly and LFSS was not, it should be noted that higher stimulus amplitudes were delivered with HFSS as compared to LFSS because of the charge-density limitations imposed. Therefore, it was not possible to precisely measure the relative sensitivity of HFSS and LFSS for direct excitation of the ganglion cell. Nevertheless, the present results suggest that (1) LFSS is much more effective at eliciting a synaptically mediated response than a response from direct activation of the ganglion cell, and (2) the response to HFSS is primarily through direct excitation of the ganglion cell and not through synaptic activation.

In general, the neuronal response to direct electric stimulation (i.e. non-synaptic component) is thought to be governed by at least two factors: first, the membrane potential of the target neuron is modulated by the electric field of the stimulus with a time course determined by the resistive and capacitive properties of the membrane and any cells or tissue between the stimulating electrode and the target neuron (Tehovnik et al., 2006). Second, the change in membrane potential will open or close voltage-gated ion channels that will, in turn, further influence the membrane potential. The expression of ion channels is heterogeneous across cell classes, cell types, and across individual neuronal substructures. In addition, the kinetics and/or activation/inactivation properties of each channel type can be different as well. This suggests that knowledge of both ion channel distributions and their corresponding response properties may be necessary to understand the neuronal response to electric stimulation.

A computational model explored the possible contribution of specific types of ion channels to the frequency-dependent responses that were observed experimentally. Previous studies have shown that voltage gated sodium channels underlie the response of ganglion cells (and axons) to direct activation while both T- and L-type calcium channels underlie the release of neurotransmitter from the presynaptic neurons (bipolar cells and photoreceptors) that lead to indirect (synaptic) activation. Thoreson, 36 Mol. Neurobiol. 205 (2007). Therefore, the model was used to determine how each of these three channels respond to the range of sinusoidal frequencies delivered experimentally.

In the model, current through L- and T-type calcium channels was maximal at low stimulus frequencies (FIG. 7g). This is consistent with the results from our physiological experiments which found that presynaptic neurons (photoreceptors and bipolar cells) were maximally activated with low frequency sinusoidal stimulation (FIG. 3). At higher stimulation frequencies, the model revealed that an L-type calcium channel responds weakly, consistent with the lack of synaptically mediated activity in ganglion cells found experimentally.

The moderate level of activity in response to high frequency stimulation of the modeled T-type calcium channel was somewhat surprising. It is possible that the small amount of synaptic activity seen experimentally in response to high frequency stimulation was mediated by T-type channels. This synaptic response was relatively weak, however, and therefore the ability of T-type channels to respond to high stimulus frequencies may be an artifact of the specific T-type channel as modeled.

The model showed that the sodium channel responded optimally to relatively high stimulus frequencies, consistent with the results from our physiological experiments which showed that direct activation of the ganglion cell can be achieved with high frequency stimulation (FIG. 3). The ability of ganglion cells to respond to such high frequencies is likely the result of the rapid activation kinetics of sodium channels. At low frequencies, the modeled sodium channel responded poorly (weak responses up to ˜40 Hz) (FIG. 7g) consistent with experimental results in which low frequency stimulation did not elicit responses via direct activation of the ganglion cell. The relatively weak response of the model sodium channel to low frequency stimulation is a result of the inactivation mechanism, causing the channel to close during the depolarizing phase of stimulus. Thus, at stimulus frequencies around 10 Hz to 25 Hz, the sodium channels that underlie the direct activation of ganglion cells (and their axons) may be inactivated, while the calcium channels that underlie the response of presynaptic bipolar cells and photoreceptors are strongly activated. Clearly, not all sodium channels are inactivated as low frequencies, otherwise the cell would not spike in response to increased excitatory input. While mechanisms to explain this discrepancy can be postulated (i.e., inactivation of a subset of sodium channels), alternatives to sodium channel inactivation at low frequencies must also be considered.

Much previous work on neural prostheses has investigated the ability of electric stimulation to elicit action potentials. See Nowak & Bullier, 118 Exp. Brain Res. 489 (1998); Tehovnik et al., 2006). As a result, such studies have focused largely on the role of voltage-gated sodium channels in the neural response to electric stimulation. Importantly, the present work suggests that voltage-gated sodium channels are not a necessary component for a neuron to respond to electric stimulation. In the physiological experiments, bipolar cells and photoreceptors were highly sensitive to LFSS, despite the fact that they are non-spiking, do not exhibit voltage-gated sodium currents (Kawai et al., 30 Neuron 451 (2001); Kawai et al., 943 Brain Res. 48 (2002)), and do not express dense regions of sodium channels (Cui & Pan, 25 Vis. Neurosci. 635 (2008)). This suggests that other types of voltage gated ion channels may underlie the response to electric stimulation in these cells; results from the computer simulation implicate voltage gated calcium channels as a likely candidate. It is likely that other types of voltage gated ion channels will also influence the response to electric stimulation.

The present results suggest that bipolar cells and photoreceptors are optimally activated at different stimulation frequencies (FIG. 6). Although the model results do not offer a definitive mechanism responsible for this difference, some inferences about factors that may contribute are as follows: First, both L- and T-type calcium channels mediate release from bipolar cells (Protti, 1998; Hu et al., 26 Vis. Neurosci. 177 (2009)), while only L-type channels mediate release in photoreceptors (Thoreson, 2007). The high sensitivity of L-type channels to low frequencies in the model is consistent with our experimental finding that photoreceptors were activated by the lowest stimulation frequencies we tested (5 Hz). Second, the synaptic terminals of several bipolar cells sub-types are thought to contain T-type channels exclusively. Pan et al., 32 Neuron 89 (2001). The model suggests that T-type channels respond optimally to low-to-mid frequencies but respond weakly to very low frequencies. This is consistent with the experimental observation that bipolar cell activation was stronger at 25 Hz than at 5 Hz. For bipolar cell terminals that do express both L- and T-type channels, however, it is not clear, why their frequency sensitivities are different from photoreceptors. It may be that photoreceptors and some bipolar cells respond to 5-Hz stimulation, but that the photoreceptor response is much stronger and overwhelms the bipolar cell response. Third, each class of ion channel contains multiple sub-types, each of which can have different kinetics. For example, three sub-types of T-type channels have been identified, and each activate and inactivate with different kinetics (Hu et al., 2009). The model contained only a single type each of L- and T-type channels, and it is possible therefore that the differences in the kinetics between the channels in our model and the actual channels present in the retina may account for the observed differences between photoreceptor and bipolar cell responses.

Other mechanisms may contribute to the frequency-dependent responses observed experimentally. For example, the resistive and capacitive properties of the tissue between the stimulating electrode and the target neuron may influence the frequency-dependence of the response (e.g., the bipolar cells and the stimulating electrode are separated by a layer of ganglion cells). Also, the membrane properties of the target neuron (e.g., its time constant) may influence the frequency response. In addition, the differential response of each class of retinal neuron to different frequencies of stimulation could arise, at least in part, from several other factors associated with synaptic release and neuronal signaling. These include the temporal relationship between internal calcium concentration and subsequent release of transmitter vesicles, desensitization of ligand-gated channels, and ion depletion and uptake kinetics. Further effort may determine the extent to which these factors influence the frequency-dependence. Because the model did not include all of the elements that could potentially modulate the frequency response, the specific frequency predictions for a given ion channel may not match precisely the physiological response. A key result from the model is that the different kinetics and distribution of ion channels influence the response sensitivity to different frequencies of electric stimulation.

Implications for use of sinusoidal stimulation in a retinal prosthetic Interestingly, the present results also suggest that the use of LFSS in retinal prostheses may reduce the need to position the stimulating electrode close to the targeted neurons. Using conventional pulsatile stimulation, stimulating electrodes must be positioned relatively close to the ganglion cell layer in order to reduce the thresholds required to elicit percepts (Jensen et al., 2003; Sekirnjak et al., 2006; Sekirnjak et al., 2008). Using LFSS, however, presynaptic neurons were highly sensitive to stimulation even at relatively large distances from the stimulating electrode (FIGS. 3, 6). In our experimental setup, photoreceptors were ˜4× farther from the stimulating electrode than ganglion cells (125 μm vs. 30 μm) and bipolar cells were ˜2× farther (75 μm vs. 30 μm). It is somewhat surprising therefore that photoreceptors were preferentially activated by 5-Hz stimulation and bipolar cells by 25-Hz stimulation since much previous work indicates that activation thresholds are inversely proportional to the square of distance from the stimulating electrode (Tehovnik et al., 2006). This suggests that the challenge of positioning the stimulating electrode extremely close to the ganglion cell layer may be less critical for success with LFSS. In the present experimental setup, the stimulating electrode was positioned on the vitreal side of the retina (epiretinal). Positioning the stimulating electrode closer to bipolar cells (e.g., subretinally or with penetrating electrodes) may further reduce the thresholds observed.

In implementing sinusoidal stimulation techniques, for example in a retinal prosthetic, several considerations can be evaluated. First, because the current work was performed on healthy retina, it may be necessary to confirm that similar results are obtained when LFSS is applied to the degenerate retina. The activation of photoreceptors at very low stimulus frequencies (5 Hz to 10 Hz) may not be useful in retinal prostheses when these cells have degenerated as a result of outer retinal diseases. Also, because LFSS targets presynaptic neurons, it may be necessary that bipolar cells remain viable and that they maintain synaptic connections with ganglion cells. These are both likely to be the case; anatomical studies have shown that bipolar cells remain largely intact (Gargini et al., 32 Neurosci. Biobehav. Rev. 378 (2007)), and physiological studies suggest that synaptic connections to ganglion cells remain functional, although the nature of these connections may vary from normal (Margolis et al., 28 J. Neurosci. 6526 (2008); Stasheff, 99 J. Neurophysiol. 1408 (2008)). Another consideration is that there are many sub-types of bipolar and ganglion cells. Masland, 4 Nat. Neurosci. 877 (2001). This raises the possibility that a particular frequency of sinusoidal stimulation may preferentially activate only a subset of bipolar or ganglion cells. The particular sub-types of neurons that are activated will likely have a corresponding effect on the elicited visual percept (e.g., activation of the magnocellular versus parvocellular pathways).

Charge density limits are another consideration prior to the implementation of sinusoidal stimulation in a neural prosthetic. A previous study using pulsatile stimulation found that the charge density at threshold was 0.093 mC/cm2 for direct activation of the ganglion cell and 0.219 mC/cm2 for activation of presynaptic neurons (Fried et al., 2006). In the present study, the charge density at threshold was relatively low for short-duration pulses (0.046 mC/cm2). For sinusoidal stimulation, however, the charge density levels at threshold were relatively high, both for HFSS (0.35 mC/cm2) and LFSS (0.49-0.51 mC/cm2). These values are slightly higher than the safe limit of charge density of 0.3 mC/cm2 widely used in similar types of studies. See Brummer & Turner, IEEE Trans. Biomed. Engin. 440 (1977); Sekirnjak et al., 2006).

There are several factors in determining how sinusoidal waveforms can be implemented safely in a neural prosthetic. Firstly, although the charge densities used here were relatively high, new electrode materials are being developed that allow higher charge densities to be safely delivered. Cogan, 10 Ann. Rev. Biomed. Engin. 275 (2008). Second, the present study involved epi-retinal stimulation where the stimulating electrode is 25 μm above the tissue, allowing a significant amount of current spread through the bathing solution. Other electrode configurations, such as sub-retinal or penetrating electrodes, may reduce the stimulus levels necessary to produce the desired response, thereby reducing the charge density levels. Finally, the appropriate charge density safety limits for sinusoidal stimulation are not known and may be different from the estimated charge density limits for pulsatile stimulation. (McCreery et al., 37 IEEE Trans. Biomed. Engin. 996 (1990).

The present invention provides for the use of sinusoidal stimulation in other types of neural prosthetics as well. The present results have important implications for DBS as well as for other types of neural prostheses. For example, DBS of the subthalamic nucleus (STN) for the treatment of Parkinson's Disease (PD) (Bejjani et al., 340 N. Eng. J. Med. 7476 (1999); Stefurak et al., 18 Mov. Disord. 1508 (2003); Parsons et al., 5 Lancet Neurol. 578 (2006)), often results in side effects, such as cognitive and mood changes, that are thought to arise from incidental activation of passing axons from nearby limbic circuits. LFSS may reduce these side effects by avoiding activation of passing axons that arise from these nearby circuits. For LFSS to be implemented it will be necessary to evaluate whether the elicited neural activity achieves similar clinical outcomes. Previous work has shown that the activation of afferent fibers projecting to the STN underlies the effectiveness of DBS for PD. Gradinaru et al., 324 Sci. 354 (2009). This raises the possibility that LFSS-mediated activation of presynaptic neurons in the STN could reproduce similar patterns of neural activity to those elicited by DBS for PD. Further support for the use of LFSS in other neural prosthetic applications comes from a recent study that used sinusoidal modulation of an electric field across the hippocampus to reduce seizures in an epileptic model of rat. Sunderam et al., 6 J. Neural Engin. 1 (2009). The mechanisms of neuronal activation were not elucidated in that study—it will be interesting to learn whether mechanisms similar to the ones we describe here underlie the reported effectiveness.

The ability to selectively target individual classes of neurons by varying stimulus frequency has considerable potential in retinal implants and neural prosthetics in general. A recent physiological study found that bipolar cells produced robust synaptic output in response to sinusoidal electric stimulation at frequencies of ≦25 Hz, but responded only weakly to 100 Hz-stimulation (Freeman et al., 2010). Therefore, it is important to understand the physiological mechanisms underlying this frequency dependence as a step towards improving methods of selective activation. Using a morphologically realistic bipolar cell model, the present work provides evidence that the preferential response of bipolar cells to low stimulus frequencies is largely due to the slow response dynamics of calcium channels, and not due to the passive electrical properties of the membrane.

Using both a two-compartment and a morphologically realistic, multi-compartment model, passive filtering by the membrane was lowpass with a relatively high cutoff frequency. The cutoff frequencies for the two-compartment and multi-compartment models were 895 Hz and 717 Hz, respectively—both significantly higher than the range of frequencies (10-25 Hz) that elicited bipolar-cell mediated synaptic responses in retinal ganglion cells. This high cutoff frequency was preserved over a wide range of membrane parameters, cell sizes, and cell morphologies. The lowest cutoff frequency observed for the passive membrane model was ˜115 Hz, and this occurred only by decreasing axonal diameter to one-half nominal (resulting diameter=0.36 μm) and simultaneously increasing axonal length to double the nominal value (resulting length=79.4 μm) (FIG. 14B). These values of axonal length and diameter are at the outer limits of those reported in anatomical studies (Euler & Wassle, 1995; Tsukamoto et al., 2001; Ghosh et al., 2004; Oltedal et al., 2009), suggesting that typical cutoff frequencies arising from the passive membrane properties of bipolar cells are likely to be significantly higher than 115 Hz.

The results of the passive membrane models (both the two-compartment and multi-compartment) are consistent with two recent modeling studies on retinal bipolar cells. One study showed that in response to extracellular stimulation with voltage steps, the rise time of membrane potential was faster in bipolar cells with shorter axons, but the steady-state values of membrane potential were lower (Gerhardt et al., 2010). This is consistent with our finding that bipolar cells with shorter axons had higher cutoff frequencies and reduced gain (FIG. 15A-15B). Another computational study modulated membrane potential at the bipolar cell soma sinusoidally (voltage-clamp) and measured the resulting membrane potential at the terminals (Oltedal et al., 2009). Their results indicated lowpass filtering as a result of the passive propagation of signals from the bipolar cell soma to the terminals. They found that the cutoff frequency increased with axonal diameter, while increasing the axonal length or intra-axonal resistivity resulted in a lower cutoff frequency. Also, they found cutoff frequencies that were relatively high, ranging from 300 Hz to 1800 Hz. These findings are consistent with the present results showing that the passive membrane properties of bipolar cells attenuate the response to extracellular stimulation only at relatively high frequencies.

An intuitive explanation as to why the cutoff frequency was highly dependent on intra-axonal resistance can be obtained by analyzing the circuit of the two-compartment model (FIG. 9B). For the nominal bipolar cell, the intra-axonal resistance (Raxon=0.27 GΩ) was much less than the membrane resistance of the soma (Rsoma=5.98 GΩ) or terminals (Rterm=27.9 GΩ). For low frequency stimulation, the impedance of the capacitors is extremely large (approaching infinite impedance for DC), and therefore the relationship between the stimulus (Vstim) and the membrane potential in the terminals (Vterm) can be approximated by a simple voltage divider between the terminal resistor (Rterm) and the other two resistors (Rsoma+Raxon). But since Raxon<<Rsoma, the effect of changing Raxon is negligible. As the stimulus frequency is increased, the impedance of the capacitors becomes smaller, leading to a smaller impedance of both the soma and terminal compartments. As a result, the relative amount of voltage dropped across Raxon becomes larger, and changes in the value of Raxon are no longer negligible. Therefore, changes in Raxon will alter the cutoff frequency of the circuit, but only for stimulation at high frequencies.

There are implications for selective activation of individual types of bipolar cells, as shown herein. Bipolar cells can be broadly categorized as either ON or OFF based on the polarity of their response to light (Werblin & Dowling, 1969). There are anatomical differences between these cell classes; ON cells have longer axonal processes and ramify within the inner portion of the inner plexiform layer (IPL), while OFF cells have shorter processes and ramify within the outer portion of the IPL (Famiglietti & Kolb, 1976). The models simulated here allowed us to explore whether the correct choice of stimulus frequency could facilitate the preferential activation of either ON or OFF bipolar cells.

The shorter axonal length of OFF bipolar cells corresponds to a lower intra-axonal resistance as compared to ON bipolar cells. This results in a higher cutoff frequency for OFF cells relative to ON cells, yielding a range of frequencies over which OFF bipolar cells could potentially be depolarized while producing little or no depolarization in ON bipolar cells (FIG. 15B). Longer axons (i.e., those in ON bipolar cells) also have a significantly larger gain than shorter axons (FIG. 15A). Therefore, high frequency stimulation (e.g., 500 Hz) could produce a response of similar magnitude in ON and OFF cells, even though the ON cell response is attenuated at this frequency. Therefore, despite having a larger cutoff frequency, it may not be possible to preferentially activate OFF cells at high frequencies. Furthermore, our results suggest that calcium channel dynamics would limit the ability to produce synaptic output for such rapid fluctuations in membrane potential (FIG. 20).

Another possibility to consider is whether ON cells can be preferentially activated for low to moderate stimulus frequencies. This is because longer axons have a higher sensitivity than shorter axons at these stimulus frequencies (FIG. 15A), and the terminals of ON bipolar cells are slightly closer to the stimulating electrode than those of OFF bipolar cells (at least for epi-retinal stimulation). Assuming that the length of the axons in ON versus OFF bipolar cells differ by a factor of two, then the results from FIG. 15A suggest that ON cells will be about ˜20% more sensitive than OFF cells. The shorter distance between the stimulating electrode and ON bipolar cell terminals may further facilitate preferential activation of ON cells, but this will depend critically on the distance between the electrode and the inner surface of the retina. For example, in chronic retinal implants, the reported distance between a given electrode and the inner retinal surface is thought to range from 100 to 1,000 μm. de Balthasar et al., 49 Invest. Ophthalmol. Vis. Sci. 23030 (2008). Because the human IPL is ˜40 μm thick (Kolb & Dekorver, 303 J. Comp. Neuro 617 (1991)), electrodes that are approximately 100 μm from the inner retinal surface would be significantly closer to the inner most portion of the IPL (and thus ON bipolar cell terminals), potentially allowing preferential activation of ON cells. In contrast, electrodes that are 1,000 μm from the retina would effectively be the same distance from the terminals of ON and OFF bipolar cells, making such preferential activation unlikely. The development of penetrating electrodes that allow electrodes to be positioned at specific depths within the retina may improve the ability to preferentially activate ON versus OFF bipolar cells. Palanker et al., 2 J. Neural Engin. 5105 (2005); Winter et al., 2007.

The expression of T- and L-type calcium channels varies across bipolar cells. While some bipolar cells display both L- and T-type currents, other express primarily L- or T-type currents. Hu et al., 26 Vis. Neurosci. 177 (2009). These differences may serve as a basis for selective activation of individual types of bipolar cells using sinusoidal stimulation. For example, T-type channels responded only to relatively low frequencies (<25 Hz), while L-type channels responded to low and moderate frequencies (cutoff frequency ranging from 65 Hz-500 Hz). Thus, in response to stimulation at 60 Hz, only the L-type channels will open and allow calcium to flow into the cell, producing synaptic release only from those bipolar cells that express L-type channels. This approach would be most beneficial if the expression of L- or T-type channels were correlated to specific physiological sub-types of bipolar cells; it is unknown whether this is the case. Awatramani & Slaughter, 2000; Euler & Masland, 2000; Hu et al., 2009. Recent work showed there is a differential expression of T-type calcium channels in ON versus OFF ganglion cells (Margolis et al., 30 J. Neurosci. 7127 (2010)), raising the possibility that cell-type specific expression patterns may also exist in bipolar cells.

The present results suggest that the relatively slow kinetics of L- and T-type calcium channels may limit the ability of bipolar cells to initiate synaptic release for rapid fluctuations in membrane potential. There are two exceptions, however, where synaptic release for high frequency stimulation may be possible. First, the cutoff frequency for L-type channels increases for larger membrane potential fluctuations. Therefore, it may be possible to elicit L-type calcium currents if the membrane potential can be modulated by relatively large amounts (i.e., beyond the normal physiological range of ˜15 to 25 mV). Nelson & Kolb, 1983; Euler & Masland, 2000. An estimate of the maximum level of membrane depolarization that is possible with extracellular stimulation has not, however, been reported. Second, although T-type channels respond optimally at low frequencies (˜5-25 Hz), our results suggest the steady-state conductance is non-zero for rapid modulations in membrane potential (FIG. 17B-17D). Therefore, high frequency stimulation may elicit currents through T-type channels even though the channels themselves do not open and close at the stimulus frequency. Importantly, this feature was not specific to the T-type model being employed since the high-frequency plateau of the frequency response was observed in both models we tested. Huguenard & McCormick, 68 J. Neurophysiol. 1373 (1992); De Schutter & Bower 1994. It will be necessary to determine if T-type channels exhibit similar behavior under physiological conditions, or whether this effect is an artifact of the equations used to describe T-type channel behavior. Also, even if it is possible to elicit L- or T-type calcium currents at relatively high stimulus frequencies, the relationship between calcium influx and vesicle release is not instantaneous, but occurs with a time constant of ˜1.1 ms. Oltedal & Hartveit, 588 J. Physiol. 1469 (2010). Therefore, the release of synaptic vesicles will be attenuated for calcium currents oscillating faster than ˜900 Hz.

The total calcium channel conductance in bipolar cells has not been reported previously. In preliminary studies, whole-cell simulations of the multi-compartment model determined the value of L-type calcium conductance that would yield currents similar in magnitude to those reported physiologically. Protti & Llano, 1998. The L-type conductance was ˜5 mS/cm2, similar in magnitude to the calcium conductance estimated in ganglion cells (˜1 mS/cm2) (Fohlmeister & Miller, 78 J. Neurophysiol. 1935 (1997)), but significantly larger than the leak conductance (0.048 mS/cm2). In simulations for L-type channels, if the calcium channel conductance was set to be larger than the leak conductance, then this resulted in a positive feedback effect: depolarization of the membrane caused L-type channels to open, and this caused more depolarization, and so on, until all channels were open and the membrane potential rested at the calcium reversal potential (ECa=+45 mV). This positive feedback effect was avoided by setting calcium channel conductance to be equal to the leak conductance—at this level the opening/closing of calcium channels did not affect the relationship between the applied stimulus and the resulting modulations in membrane potential (FIG. 19B). Note that such positive feedback was not a concern for T-type channels because these channels inactivate at depolarized potentials, allowing the resting potential to return to the leakage reversal potential (Eleak).

Although regenerative activity of voltage-gated calcium channels has been shown to produce depolarization in bipolar cells (Protti et al., 2000; Ma & Pan, 20 Vis. Neurosci. 131 (2003)), the membrane potential is quickly returned to rest (−40 to −50 mV) as a result of the activation of other voltage-gated ion channels (e.g., potassium). Protti et al., 2000. Other voltage-gated channels in the multi-compartment model were not incorporated for two reasons. First, the inclusion of such channels would make it difficult to separate the effects of passive membrane filtering and calcium channel dynamics from those of other channels. In particular, the continual opening and closing of both voltage-gated sodium and potassium channels would alter membrane conductance, and this would affect the relationship between the applied stimulus and the bipolar cell membrane potential. Second, the expression pattern of these other channels across different types of bipolar cells is not fully understood. For example, voltage-dependent potassium currents have been found to differ between rod bipolar and cone bipolar cells, as well as between different types of cone bipolar cells. Hu & Pan, 19 Vis. Neurosci. 163 (2002). Similarly, voltage-gated sodium currents have been reported, but only in a subset of bipolar cells. Pan & Hu, 84 J. Neurophysiol. 2564 (2000). Therefore, inclusion of these channels into the model would require new assumptions as to the types and densities of these channels.

The present embodiments provide for implications for temporal resolution of prosthetic vision. Ganglion cell spiking can be elicited through activation of presynaptic bipolar cells; this is referred to as indirect activation. In response to repetitive stimulation with pulses, the ganglion cell response to the first pulse is robust, but the responses to subsequent pulses are greatly desensitized. Jensen & Rizzo, 4 J. Neural Engin. S1 (2007); Freeman & Fried, 2011. Such desensitization has been reported for pulse rates as low as 2 Hz and severely limits the ability to control the temporal pattern of ganglion cell spiking elicited through the synaptic network. The present results suggest that L- and T-type calcium channels can respond to frequencies of tens or hundreds of hertz (FIGS. 16-17). Therefore, it is unlikely that calcium channels are responsible for the desensitization observed physiologically. If such a desensitization mechanism could be avoided, then it is possible the indirect response of ganglion cells will be limited by the slow kinetics of L- and T-type calcium channels at high stimulus frequencies.

The present invention also provides for prosthetic devices that deliver the low-frequency sinoid(s) to the target neurons. For example, a low-frequency sinoid emitter can be incorporated into a visual apparatus for creation of artificial vision. See, e.g., U.S. Pat. No. 8,000,000. Additionally or alternatively, the prosthetic can be used in the brain for treating neurological conditions as exemplified herein. See, e.g., U.S. Pat. No. 6,591,138; No. 6,690,974; No. 7,894,905; U.S. Patent Appl. Publications No. 2009/0246140; No. 2009/0112279; No. 2009/0069863; No. 2010/0217341.

Thus, for example, the present invention provides for system for treating a neurological disorder in a human patient, the system comprising a control module (which may be implantable) including electronic circuitry, and at least one electrode connected to the electronic circuitry, wherein the electrode is adapted to be placed on, near, or in the patient's brain, wherein the electronic circuitry of the control module is adapted to selectively stimulate the patient's neuronal cells with a sinusoidal electrical signal having a frequency of about 100 Hz or less. The frequency can be about 50 Hz, 25 Hz, 10 Hz, or 5 Hz. The frequency can be about 25 Hz or less, between 5 Hz and 25 Hz (inclusive), or between about 10 Hz to about 25 Hz (inclusive).

Another aspect of the invention provides for a method for treating a neurological disorders comprising implanting a stimulation electrode in, on, or near the brain of a patient; providing a control module (e.g., by implanting in the patient); and causing the control module to apply a low-frequency sinusoidal stimulation signal to the stimulation electrode, wherein the low-frequency stimulation signal has a fundamental frequency below approximately 100 Hz. The frequency can be about 50 Hz, 25 Hz, 10 Hz, or 5 Hz. The frequency can be about 25 Hz or less, between 5 Hz and 25 Hz (inclusive), or between about 10 Hz to about 25 Hz (inclusive). In these devices, as constructed for the purposes described herein, low frequency sine waves can restrict activation to a narrow region around the electrode because sodium channels, which are found in axons, do not respond to low frequencies.

The selective activation as provided herein can be used to alleviate or treat a neurological condition such as neurologically-mediated cardiac and cardiovascular disorders, headache disorders (including migraine), inadequate cerebral perfusion, movement disorders, neurodegenerative disorders, pain, psychiatric and mood disorders, seizure disorders (such as epilepsy), spinal cord disorders, vision disorders, and voiding disorders.

Further aspects provide for the selective stimulation according to the present invention in combination with other therapy directed to the particular indication. Thus, for example, when the disorder is Parkinson's disease, therapy may include use of the present invention in combination with stem cell therapy, physical therapy, and/or drug therapy (such as levodopa).

### EXAMPLES

### Example 1

### Animal Preparation and Retina Isolation

The care and use of animals followed all federal and institutional guidelines, and all protocols were approved by the Institutional Animal Care and Use Committees of the Boston VA Healthcare System and/or the Subcommittee of Research Animal Care of the Massachusetts General Hospital. New Zealand White Rabbits (˜2.5 kg) were anesthetized with injections of xylazine/ketamine and subsequently euthanized with an intracardial injection of pentobarbital sodium. Immediately after death, the eyes were removed. All procedures following eye removal were performed under dim red illumination. The front of the eye was removed, the vitreous was eliminated. The retina was separated from the retinal pigment epithelium and mounted, photoreceptor side down, to a 10-mm square piece of Millipore filter paper (0.45 μm HA Membrane Filter) that was mounted with vacuum grease to the recording chamber (˜1.0 ml volume). A 2-mm circle in the center of the Millipore paper allowed light from below to be projected on to the photoreceptors.

### Example 2

### Electrophysiology and Light Responses

Patch pipettes were used to make small holes in the inner limiting membrane, and ganglion cells with large somata were targeted under visual control. Spiking was recorded with a cell-attached patch electrode (4-8MΩ) filled with superfusate. For whole-cell recordings, the patch electrode was filled with (in mM): 113 CsMeSO₄, 1 MgSO₄, 7.8×10⁻³ CaCl₂, 0.1 BABTA, 10 HEPES, 4 ATP-Na₂, 0.5 GTP-Na₃, 5 lidocaine N-ethyl bromide (QX314-BR), 7.5 neurobiotin chloride, pH 7.2. Excitatory currents were revealed by clamping at −60 mV (ECl). Two silver-chloride coated silver wires served as the ground and were positioned at opposite edges of the recording chamber each approximately 15 mm from the targeted cell. The retina was continuously perfused at 4 mL/min with Ames' (pH 7.4) at 36° C., equilibrated with 95% O₂ and 5% CO₂. Pharmacological agents were applied to the bath by switching a 3-way stopcock to a 200 mL reservoir of Ames' containing one or more of the following blockers: 50 μM 6-cyano-7-nitroquinoxaline-2,3-dione (CNQX), 100 μM cadmium chloride (CdCl₂).

The light stimulus was controlled by VisionWorks software, and data acquisition and stimulus triggering was controlled by custom software written in LabView (National Instruments) and Matlab (Mathworks). Light stimuli were projected on to the retina from below through an LCD projector (InFocus) and focused onto the photoreceptor outer segments with a steady, photopic background. Light stimuli consisted of stationary flashed squares (size range: 100-1000 μm), 1-sec duration, centered at the soma. Stimulus intensity was 50-75% above background light level. Other than noting whether targeted ganglion cells were ON or OFF, they were not further classified.

### Example 3

### Electric Stimulation

Electric stimulation was delivered via a 10 kΩ Platinum-Iridium electrode (MicroProbes); the exposed area was conical with an approximate height of 125 μm and base diameter of 15 μm, giving a surface area of ˜5,900 μm², comparable to a 40 μm disk electrode. Pulse and sinusoidal stimuli were controlled by Multi-Channel Systems STG2004 hardware and software. Two silver-chloride coated silver wires served as the return; each was positioned approximately 8 mm from the targeted cell and approximately 12 mm from each other. The height of the stimulating electrode remained fixed at 25 μm above the inner limiting membrane. The stimulating electrode was placed either directly over the sodium-channel band on the proximal axon, or ˜1 mm lateral to the soma directly over the distal axon. Because of the use of patch clamp, spikes were clearly visible through the stimulus artifact. The efficacy of various stimulation waveforms (0.2-ms pulses and 5-Hz to 100-Hz sinusoids) was tested for the two different electrode positions.

### Example 4

### Location of the Sodium-Channel Band

In response to short-duration pulses, the location of the sodium-channel band has been shown to correspond to the center of the region with the lowest threshold and is generally centered between 20 and 60 μm from the soma along the proximal axon (Fried et al., 2009). Using an iterative process, the center of the low-threshold region was found quickly: movement of the stimulating electrode towards the center of the low-threshold region resulted in decreasing thresholds while movement away from the center resulted in increasing thresholds. This location was used as the approximate center of the sodium-channel band. Preliminary testing indicated that thresholds for sinusoidal stimulation were also lowest over the sodium-channel band (FIG. 1).

### Example 5

### Location of the Distal Axon

The trajectory of the distal axon was ascertained by studying the pattern of thresholds in response to rectangular pulses of electric stimulation. During the dissection of the retina, the location of the optic disk was noted and the tissue oriented so that axons generally coursed in a constant direction (from right to left in this preparation). Electric pulse stimulation was used to more precisely define the axon location. A typical search algorithm placed the stimulating electrode 100 μm left of the soma and then delivered a series of ten increasing-amplitude pulses. If the pulses elicited spikes, the stimulating electrode was moved perpendicular to the presumed axon trajectory in 10 μm steps to find the location at which the lowest pulse amplitudes could elicit spikes. This was considered to be the axon location. The stimulating electrode was then moved an additional 100 μm to the left and the process repeated until the axon position was determined at a distance of ˜1000 μm from the soma.

### Example 6

### Rectangular Pulses

Pulsatile stimuli were biphasic pulses (equal and opposite rectangular phases) delivered at 10 pulses per second (phase duration: 200 μsec; interphase delay: 10 ms; cathodic phase first). The interphase delay was long enough for the neural response to the cathodic pulse to be completed before the onset of the anodic phase. For each stimulus amplitude, 15-30 pulses were delivered and there was a delay of >5 sec between stimulation epochs. Pulses of this duration and over the range of stimulus amplitudes produced either a single spike or no spike. If a spike was elicited, it immediately followed the cathodal pulse. Therefore, the number of pulses that elicited a spike was normalized to the total number of pulses delivered to give the fraction of pulses that elicited spikes.

### Example 7

### Sinusoidal Waveforms

Sinusoidal waveforms were delivered at frequencies of 5, 10, 25, and 100 Hz. Sinusoidal stimuli were delivered for one second, using a linear onset and offset ramp of 40 ms to reduce the spectral splatter induced by sudden stimulus onset/offset. Because a typical cell was held for <30 min and there were several stimulus conditions to be tested on a given cell, time constrains limited the number of stimulus presentations; each stimulus amplitude was delivered once, with a delay of at least 5 sec between consecutive stimuli. An array of stimulus amplitudes were delivered in steps of 1-2 μA, where the amplitudes were chosen with the goal of covering the full dynamic range of the neuron. For each cell, the order of presentation for the various stimulus waveforms was randomized. The maximum amplitude for which the charge density of the stimulating electrode remained below safe limits was estimated using a method described previously (Brummer & Turner, 1977): the stimulus amplitude was increased until microscopic bubbles were seen to form on the electrode tip.

Based on these results, the maximum stimulation levels were set at: 4 μA, 9 μA, 18 μA, and 36 μA for 5 Hz, 10 Hz, 25 Hz, and 100 Hz, respectively. For pulses, the stimulus level that exceeded charge density limits was not estimated since a threshold response was always achieved below this stimulus level. Because sinusoidal stimulation typically elicited multiple spikes per stimulus period, we plotted the number of spikes elicited by the one second stimulus as a function of stimulus amplitude. This is a different measurement than the probability curves used for pulsatile stimulation, and this should be taken into account when comparing data from pulsatile and sinusoidal stimulation. Stimulus amplitude was reported in terms of current levels (μA) instead of charge per phase (nanocoulombs/phase) to facilitate comparison across stimulus frequencies (charge/phase varies considerably across the frequencies tested).

### Example 8

### Stimulus Threshold and Statistical Tests

The cells used in this study did not exhibit spontaneous firing and therefore all recorded spikes were assumed to be stimulus induced. The number of spikes (R) was measured for a range of stimulus amplitudes (S) in steps of 1-2 μA, and sigmoidal curves were found to fit the data well (<r²>=0.913±0.097), using the equation: R=A*Sⁿ/(Sⁿ+σⁿ), where A is the saturation level, σ is the input current required to reach half of saturation, and n is the order of the sigmoid. Stimulus threshold was therefore defined as the stimulus amplitude necessary to produce the number of spikes equal to half the number of stimulus periods (e.g., for a 100-Hz, 1-sec sinusoidal stimulus, the stimulus level required to elicit 50 spikes is defined as threshold). Due to the limits on stimulus levels for sinusoidal stimulation, saturation level could not be reached in many cells and σ could not be used to define threshold. If a cell did not elicit a threshold number of spikes for the highest stimulus amplitude tested (as determined by the amplitude levels at which micro-bubbles were produced), the highest stimulus amplitude tested was taken to be threshold. For pulses, threshold was defined as the stimulus level necessary to elicit a spike on half the number of pulses delivered, as estimated by the best-fit sigmoidal curve. All tests for statistical significance are paired t-tests using a significance level of 5% (a=0.05).

### Example 9

### Computational Modeling

Models of a voltage-gated sodium channel and an L-type calcium channel were developed from previous physiology and modeling studies of retinal ganglion cells. Huang, 1998; Benison et al., 2001. T-type calcium channels in retinal neurons have been characterized physiologically, but an explicit model of these channels in the retina has not been developed. Therefore, model equations were based on work from cerebellar Purkinje neurons (Schutter, 1994), which have similar physiological properties as the T-type calcium channels in retinal bipolar cells (Hu, 2009). The voltage across the channels was varied sinusoidally or stepwise and the resulting sodium and calcium currents were calculated. Currents took on the general form of:

I_{Na}=g_{Na}m³h(V−E_{Na})

I_{CaL}=g_{Ca}n²(V−E_{Ca})

I_{CaT}=g_{CaT}kq(V−E_{Ca})

where g_{Na}=150 nS, g_{CaL}=2.0 nS, g_{CaT}=1.0 nS, E_{Na}=75 mV, and E_{Ca}=45 mV. The gating parameters were calculated with the equation:

dp/dt=αₚ(V)(1−p)−βp(V)p

where p=m, h, n, k, and q. The gating parameters m, h, and n are activating (open in response to depolarization), and the parameters h and q are inactivating (open in response to hyperpolarization). The functions αₚ (V) and βₚ (V) can be found in Benison et al. 2001 for I_{Na} and I_{CaL} and Schutter & Bower (1994); Schutter (1994) for I_{CaT}. Differential equations were solved in Matlab using Euler's method with a timestep of 0.01 ms.

### Example 10

### Two-Compartment and Multi-Compartment Models

Retinal bipolar cells receive synaptic input from photoreceptors in the outer retina and provide synaptic input to amacrine and ganglion cells in the inner retina. Under normal physiological conditions, fluctuations in membrane potential at the soma propagate passively down the axon to the terminals (FIG. 9A), where synaptic release is initiated. A two-compartment model of a bipolar cell modified from previous work was implemented. Mennerick et al., 78 J. Neurolphysiol. 51 (1997); Oltedal et al., 97 J. Neurophysiol. 1171 (2007). The soma and terminals were each represented by a single compartment that contained a resistor and capacitor in parallel (FIG. 9B). The two compartments were connected by a single resistor (Rₐₓₒₙ), representing resistance to current flow along the inside of the axon. Extracellular stimulation was modeled as a voltage source applied across the soma and terminal regions (Vₛₜᵢₘ). This was based on a common model of extracellular stimulation where a spatial gradient in voltage along the outside of the cell causes current to flow through and along the cell membrane. McNeal, 23 IEEE Trans. Biomed. Engin. 329 (1976). Because synaptic release is mediated by calcium entry through voltage-gated channels in the synaptic terminals, we were interested in how the membrane potential in the terminals (Vₜₑᵣₘ) varied in response to sinusoidal modulations of Vₛₜᵢₘ. The motivation for using this simple two-compartment model is that it allows linear circuit analysis to be used to derive a direct mathematical relationship between the applied stimulus (Vₛₜᵢₘ) and the membrane potential in the terminals (Vₜₑᵣₘ). Analysis was performed using Matlab software (Mathworks, Natick, Mass.).

In addition to the two-compartment model, a multi-compartment bipolar cell model developed in previous work was implemented (FIG. 12A-12B). Oltedal et al., 2009; Oltedal & Hartveit, 2010. This model was based on the morphologically reconstructed rod bipolar cell shown in FIG. 9A, and contained a total of 92 compartments. Oltedal et al., 2009. The model was implemented in the NEURON (Hines, Neural Sys: Anal. & Modeling (Kluwer, Norwell, Mass., 1993) simulation environment and modified to include the effects of extracellular electric stimulation. As with previous work implementing extracellular stimulation of a model neuron (Greenberg et al., 1999), an ideal monopolar point source was used to represent the stimulating electrode. The point source was positioned 40 μm from the terminals (i.e., 40 μm from the leftmost point of the cell in FIG. 12A), unless stated otherwise. The external medium in which the current travels was assumed to be homogeneous and infinite. The extracellular potential at each point in space is related instantaneously to the applied stimulus voltage, where extracellular potential falls inversely with distance from the point of stimulation according to the following equation:

Vₑ=(ρₑIₛₜᵢₘ)/(4πr)

where Vₑ is the extracellular potential, Istim is the amplitude of the stimulus, ρe is the resistivity of the extracellular medium (set to 110 Ωcm) (Coleman & Miller, 61 J. Neurophysiol. 218 (1989)), and r is the distance between the stimulating electrode and the center of each compartment. For each simulation, the extracellular voltage for each compartment was modulated sinusoidally and the resulting membrane potential of each compartment was determined. Non-uniformities in the electric field arising from the presence of the model cell were ignored.

For the multi-compartment model, the cell was considered as three sections: the soma, axon, and terminals. Dendrites arising from the soma were considered as part of the soma section and were not modeled separately. The following parameter values were derived from the multi-compartment model in Oltedal et al., 2009. The axon length was 39.4 μm, as measured from the soma to the first bifurcation, beyond which was considered the terminal. The axonal diameter, averaged across the length of the axon, was 0.71 μm. Specific membrane capacitance (Cₘ) was set to 1.07 μF/cm², specific membrane conductance (gₗₑₐₖ) was set to 48.00 μS/cm², and the leak reversal potential (Eₗₑₐₖ) was set to −50 mV. For a given compartment, the leak conductance and membrane capacitance was determined by scaling the specific membrane conductance and capacitance by the surface area of the membrane. The resistance to current flow along the length of the cell was modeled with a resistor connecting each compartment within the interior of the cell. For consistency, this resistor was quantified in terms of conductance (gᵢₙₜᵣₐ), and this was computed as a function of intra-cellular resistivity (ρᵢ=189.65 Ωcm, unless stated otherwise), the cross-sectional area of the cell, and the length of each compartment.

For the two-compartment model, the nominal values of the resistors and capacitors in the soma and terminals were derived from values in the multi-compartment model by scaling specific membrane conductance (48.0 μS/cm2) and capacitance (1.07 μF/cm2) to the area of the soma (348.3 μm2) and terminal (74.7 μm2) regions. The resulting values of the resistors and capacitors were: Rsoma=5.98 GΩ, Csoma=3.7 pF, Rterm=27.9 GΩ, Cterm=0.8 pF. The axonal resistance was computed by summing up the intra-axonal resistance along the length of the multi-compartment neuron giving a value of Raxon=272.2 MΩ.

The anatomical properties of bipolar cells can vary considerably across the ˜10 types of bipolar cells. Euler & Wassle, 1995; Boycott & Wassle, 1999; Wu et al., 2000. Interest in understanding the sensitivity of the model to changes in bipolar cell anatomy, including variations in axonal length and diameter, as well as soma and terminal size, required definition of a range of values over which each parameter was varied. For example, axonal length varies from 10 to 50 μm across bipolar cell types (Euler & Wassle, 1995; Ghosh et al., J. Comp. Neurol. 70 (2004)), and other anatomical parameters, such as soma and terminal size, can vary considerably across species even in cells of the same type (Caminos et al., 56 Brain Behav. Evol. 330 (2000)). Therefore, instead of trying to replicate the precise range of configurations seen across species and across bipolar cell types within a given species, we chose to increase and decrease each parameter by a factor of 2 from nominal (39.6 μm) (total range of a factor of 4), thereby allowing characterization of the sensitivity of the model to each parameter.

Regarding normalization, for the two-compartment model, an analytical expression was derived for the transfer function, defined as Vterm/Vstim. The transfer function of the multi-compartment model, defined as Vterm/Istim, was too complex to express analytically, and therefore the frequency response was obtained by measuring Vterm in response to sinusoidal modulations of Istim as a function of stimulus frequency. The normalization procedure for the frequency response (or transfer function) contained two steps. First, the frequency response (or transfer function) obtained for nominal model parameters was normalized to unity (FIGS. 9C and 12C). Second, the frequency response (or transfer function) obtained for other model parameters was normalized by the same factor in order to allow direct comparison to nominal curves (e.g., FIG. 11A). Note that for two-compartment and multi-compartment passive models (i.e., when no calcium channels were present), the circuit contained only linear elements. Therefore, the shape of the frequency response was not dependent on stimulus amplitude, and was obtained only for a single stimulus amplitude. In some instances, all curves on in a given plot were normalized to unity to allow comparison; in these cases the axes were labeled as ‘normalized’ (e.g., FIG. 15B). Cutoff frequency is defined as the frequency at which the response is decreased from maximum by 3 dB (1/12, or 0.707 of maximum).

### Example 11

### Calcium Channel Simulations

Current flowing through L- and T-type calcium channels and into the cell initiates synaptic release from bipolar cell terminals. Tachibana, 1993; Pan et al., 2001. As a result, the dynamics of the opening/closing of L- and T-type channels in response to changes in membrane potential may play an important role in shaping the frequency response of synaptic release in response to extracellular stimulation. Therefore, we examined the gating equations for L- and T-type channels in order to investigate their contribution to the bipolar cell response independent from the effects of the passive membrane properties of the neuron.

Equations describing the voltage-dependence of these channels have not been reported in bipolar cells. Therefore, we used equations for the L-type calcium channel derived from work in retinal ganglion cells (Benison et al., 2001). This model was chosen because it exhibited similar response kinetics and threshold for activation as the physiologically reported L-type currents in bipolar cells. Tachibana, 1993; von Gersdorff & Matthews, 16 J. Neurosci. 115 (1996); Hartveit, 81 J. Neurophysiol. 2923 (1999); Hu et al., 2009. For T-type channels, we implemented a model based on cerebellar Purkinje neurons (De Schutter & Bower, 1994); these channels exhibited a relatively low threshold for activation that is characteristic of T-type currents reported from physiological studies on bipolar cells (Kaneko et al., 410 J. Physiol. 613 (1989); Hu et al., 2009). In order to test whether the results were specific to the choice of model, we also simulated other model equations for L-type (McCormick & Huguenard, 68 J. Neurophysiol. 1384 (1992)) and T-type (Huguenard & McCormick, 1992) channels based on thalamic relay neurons.

These equations were simulated in voltage-clamp conditions in which the voltage was varied sinusoidally and the resulting calcium current was measured. The voltage was oscillated about a baseline level of −50 mV; this value is approximately midway between the reported resting membrane potential of cone bipolar cells (−57.6 mV) and rod bipolar cells (−45.4 mV). Ma et al., 2005. The maximal fluctuation in bipolar cell membrane potential elicited by electric stimulation is unknown. Therefore, we tested over a wide range of voltage fluctuations, ranging from 2.5 mV to 100 mV (i.e., reaching depolarization levels of −47.5 mV to +50 mV). The L-type currents (I_{L}) and the T-type currents (I_{T}) were computed as follows:

I_{L}=g_{L}(V−E_{Ca})

I_{T}=g_{T}(V−E_{Ca})

The conductance of each channel was nonlinear, defined as:

g_{L}=g_{Lmax}m²

g_{T}=g_{Tmax}nh

where E_{Ca}=45 mV, g_{Lmax}=g_{Tmax}=gₗₑₐₖ=48.0 μS/cm², and m, n, and h are defined below. Note that the magnitude of g_{Lmax} and g_{Tmax} will not affect the shape of the frequency responses and will only scale the magnitudes of the resulting currents.

The relationship between voltage and channel conductance was based on the formalism of Hodgkin and Huxley (1952):

$$\frac{d p}{d t} = {α}_{p} ( V ) ( 1 - p ) - {β}_{p} ( V ) p$$

where p=m, n, and h. The gating parameters m and n are activating (open in response to depolarization), and the parameter h is inactivating (open in response to hyperpolarization). The voltage dependent equations αₚ (V) and βₚ (V) can be found in the original articles. De Schutter & Bower, 1994; Benison et al., 2001. Differential equations were solved in Matlab using Euler's method with a timestep of 0.01-0.1 ms. The resulting currents were measured as peak-to-peak.

### Example 12

### Incorporating Calcium Channels into the Multi-Compartment Model

Following the analysis of the multi-compartment model with only passive membrane elements, L- and T-type calcium channels were added to the terminal region of the bipolar cell in parallel with the leak conductance. The current through these channels was measured in response to sinusoidal extracellular stimulation. Since the release of synaptic vesicles results from the influx of calcium to the cell, the amount of current through the calcium channels was interpreted as a measure of synaptic release from the bipolar cell in response to electric stimulation.

The total membrane conductance of either L- or T-type calcium channels in bipolar cells has not been reported. We set the maximum membrane conductance for L- and T-type calcium channels (g_{Lmax} and g_{Tmax}) to be equal to the leak conductance (gₗₑₐₖ=g_{Lmax}=g_{Tmax}). The reason for this was that if the calcium conductance was set larger than the leak, then a regenerative response could occur where all calcium channels open and remain open; such behavior is not thought to occur under normal physiological conditions.

We claim:

1. A method of selectively activating synaptically mediated responses in ganglion cells without activating passing axons, comprising contacting a focal region around said cells with an electrode that stimulates using low-frequency sinusoidal electric signal.

2. The method of claim 1, wherein the low-frequency sinusoidal stimulation has a frequency of ≦25 Hz.

3. A method of selectively activating cells comprising exposing said cells to a low frequency sinusoidal electric signal of about ≦100 Hz.

4. The method of claim 3, wherein the cells are ganglion cells and the electric stimulus is about ≦100 Hz.

5. The method of claim 3, wherein the cells are photoreceptor cells and the electric stimulus is about 5 Hz.

6. The method of claim 3, wherein the cells are bipolar cells and the electric stimulus is about 25 Hz.
